(19) United States
(12) Patent Application Publication
(10) Pub. No.: US 2024/0351965 A1
(43) Pub. Date: Oct. 24, 2024

(54) Synthesis and Evaluation of Novel (4-Hydroxyphenyl) Substituted Carbocycles as Potent and Selective Estrogen Receptor Beta Agonists

(71) Applicants: Marquette University, Milwaukee, WI (US); Concordia University, Inc., Mequon, WI (US)

(72) Inventors: William A. Donaldson, Wauwatosa, WI (US); Daniel S. Sem, New Berlin, WI (US); Edward A. Wetzel, Big Bend, WI (US)

(21) Appl. No.: 18/685,690

(22) PCT Filed: Aug. 23, 2022
(86) PCT No.: PCT/US2022/041253

§ 371 Date: Feb. 22, 2024

Related U.S. Application Data
(60) Provisional application No. 63/236,145, filed on Aug. 23, 2021.

(51) Int. Cl. C07C 39/17; A61K 31/047; C07C 39/14

(52) U.S. Cl. CPC C07C 39/17; A61K 31/047; C07C 39/14; C07C 2602/50; C07C 2603/74

(57) ABSTRACT

Disclosed are 4-hydroxylphenyl substituted carbocycles and there use as selective agonists of the estrogen receptor beta isoform (ERβ). The disclosed compounds may be formulated as pharmaceutical compositions and administered to treat diseases associated with ER activity, such as proliferative diseases and disorders and/or psychiatric diseases or disorders.

### CROSS REFERENCE TO RELATED PATENT APPLICATIONS

The present application claims the benefit of priority to U.S. Provisional Patent Application No. 63/236,145, filed Aug. 23, 2021, the contents of which is incorporated herein by reference in its entirety.

### STATEMENT REGARDING FEDERALLY SPONSORED RESEARCH OR DEVELOPMENT

This invention was made with government support under Grant No. R15GM118304 awarded by the National Institutes of General Medical Sciences. The Government has certain rights in this invention.

### BACKGROUND

The field of the invention relates to compounds that function as agonists of estrogen receptors (ERs). In particular, the field of the invention relates to (4-hydroxyphenyl) substituted carbocycles that are specific agonists for the estrogen receptor beta (ERβ) and the use of such compounds in pharmaceutical compositions for treating diseases and disorders associated with ER activity.

Estrogens are important regulators of many physiological processes that include reproduction, cognition, cardiovascular health, and bone metabolism. (See, e.g., Deroo et al., “Estrogen Receptors and Human Disease,” J. Clin. Invest. 116:561-570(2006). Based on their widespread role in a number of physiological processes, estrogens have been implicated in a number of diseases and disorders which include cell proliferative diseases and disorders (e.g., breast cancer, ovarian cancer, endometrial cancer, colorectal cancer, and prostate cancer), neurodegenerative diseases and disorders, cardiovascular disease, and osteoporosis to name a few. (See id.). In many of these diseases and disorders, estrogen mediates its effects through the estrogen receptors (ERs).

The ERs exist in 2 main forms, ERα and ERβ, which have different tissue expression patterns. (See Mueller et al. (2001), “Estrogen receptors and endocrine diseases: lessons from estrogen receptor knockout mice,” Curr. Opin. Pharmacol. 1: 613-619). ERα and ERβ are encoded by separate genes, ESR1 and ESR2, respectively, found at different chromosomal locations, and numerous mRNA splice variants exist for both ERα and ERβ. (See, e.g., Hernyk et al., “Estrogen receptor mutations in human disease,” (2004) Endocr. Rev. 25:869-898). Because of their role in estrogen-related diseases, ERα and ERβ have been targeted for development of specific ligands that modulate their activities. The ligand specificity of ERα and ERβ differ, and a ligand that binds and functions as an agonist or antagonist for ERα may or may not bind and function as an agonist or antagonist for ERβ.

One group of ligands for ERs that have been developed are the so-called “selective estrogen receptor modulators” or “SERMs” which include tamoxifen and raloxifene. Tamoxifen and raloxifene have been observed to exhibit tissue-specific estrogenic activity. For example, tamoxifen is an antagonist in the breast and has been a safe and effective adjuvant endocrine therapy for breast cancer for almost 20 years, but tamoxifen is an ER agonist in bone and uterus. (See, e.g., Deroo et al., “Estrogen Receptors and Human Disease,” J. Clin. Invest. 116:561-570 (2006)). Raloxifene exhibits greater agonist activity in bone and less agonist activity in the uterus. (See Fabian et al., “Selective estrogen-receptor modulators for primary prevention of breast cancer,” J. Clin. Oncol. 23:1644-1655 (2005)). Whether a ligand is an ER agonist or antagonist in a particular tissue depends on several factors, including which form of the estrogen receptor predominates in the particular tissue, in other words ERα or ERβ, where the ligand may exhibit different binding affinity and/or agonist/antagonist activity for ERα versus ERβ.

ERα and ERβ agonists have a wide range of biological effects that implicate disease such as cancer and disorders of the central nervous system (CNS). Clinical studies have indicated that administering estradiol (E2) in post-menopausal hormone replacement therapy (HRT) can lead to increased incidence of breast and endometrial cancer. (See Beral et al., “Breast cancer and hormone-replacement therapy in the Million Women Study,” Lancet. 2003; 362(9382:419-27. Epub 2003/08/21. PubMed PMID: 12927427; Gann et al., “Combined hormone therapy and breast cancer: a single-edged sword,” JAMA: the journal of the American Medical Association. United States 2003. p. 3304-6; Li et al., “Relationship between long durations and different regimens of hormone therapy and risk of breast cancer,” JAMA: the Journal of the American Medical Association. 2003; 289(24):3254-63. Epub 2003/06/26. doi: 10.1001/jama.289.24.3254. PubMed PMID: 12824206; and Anderson et al., “Effects of conjugated equine estrogen in postmenopausal women with hysterectomy: the Women's Health Initiative randomized controlled trial,” JAMA: the journal of the American Medical Association. 2004; 291(14):1701-12. Epub 2004/04/15. doi: 10.1001/jama.291.14.1701. PubMed PMID: 15082697). This effect is mediated predominantly by ERα, the dominant isoform present in the mammary gland and uterus. (See Song et al., “Estrogen receptor-beta agonist diarylpropionitrile counteracts the estrogenic activity of estrogen receptor-alpha agonist propylpyrazole-triol in the mammary gland of ovariectomized Sprague Dawley rats. The Journal of steroid biochemistry and molecular biology. 2012; 130(1-2):26-35. Epub 2012/01/24. doi: 10.1016/j.jsbmb.2011.12.018. PubMed PMID: 22266284).

The increased cancer risk has led to decreased usage of HRT in post-menopausal women. But, studies also have shown that HRT can provide a positive effect mediated primarily by ERβ, which is a decrease in the risk of dementia in post-menopausal women. (See Leblanc et al., “U.S. Preventive Services Task Force Evidence Syntheses, formerly Systematic Evidence Reviews. Hormone Replacement Therapy and Cognition. Rockville (MD): Agency for Healthcare Research and Quality (US); 2002). As such, specific ERβ agonists can provide the CNS benefits of E2 with minimal side effects. However, current SERMs such as tamoxifen and raloxifene, are not specific for ERβ, have carcinogenic side effects, and provide little memory enhancement. (See Yaffe et al., “Cognitive function in postmenopausal women treated with raloxifene. New England Journal of Medicine. 2001; 344:1207-13; and Paganini-Hill et al., “Preliminary assessment of cognitive function in breast cancer patients treated with tamoxifen. Breast Cancer Research and Treatment. 2000; 64:165-76). Safer and more effective treatments can be developed by selectively targeting ER.

Thus, new ligands for estrogen receptors are desirable. In particular, new ligands that exhibit selective agonist or antagonist activity for ERβ versus ERα are desirable. These new ligands should be suitable for treating diseases and disorders associated with ER activity, such as cell proliferative diseases and disorders, psychiatric diseases and disorders, or vasomotor diseases and disorders. Such new ligands are disclosed herein in the form of (4-hydroxyphenyl) substituted carbocycles.

### SUMMARY

Disclosed are 4-hydroxylphenyl substituted carbocycles and their use as selective agonists of the estrogen receptor beta (ERβ). The disclosed compounds may be formulated as pharmaceutical compositions and administered to treat diseases associated with ER activity.

The disclosed compounds may include an optionally substituted cyclic, spirocyclic, fused, or bridged ring system bound to an optionally substituted phenol group (e.g., at a para-position (4-position) of the optionally substituted phenol group) which phenol group optionally is hydroxyl-protected. The spirocyclic ring system may include an optionally substituted spiro[5.3]nonane group:

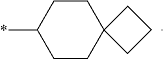 [SMILES: *C1CCC2(CCC2)CC1 ; "."]

The fused ring system may include an optionally substituted bicyclo[3.3.0]octane group, an optionally substituted bicyclo[3.3.0]octene group, an optionally substituted bicyclo[3.1.0]hexane group:

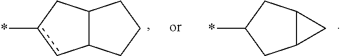

The bridged ring system may include an optionally substituted adamantyl group, an optionally substituted bicyclo[3.3.1]nonane group, or an optionally substituted bicyclo[3.3.1]nonene group:

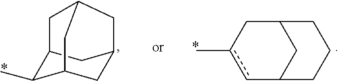

The cyclic ring system may include an optionally substituted cyclohexyl group.

In some embodiments, the disclosed compounds may have a Formula I:

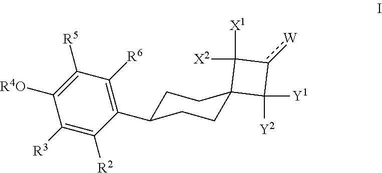

wherein
X¹, X², Y¹, and Y² are independently selected from the group consisting of hydrogen, halogen, and hydroxyl;
optionally with the proviso that when X¹ and X² are halogen then Y¹ and Y² are hydrogen and
optionally with the proviso that when Y¹ and Y² are halogen then X¹ and X² are hydrogen;
W is selected from the group consisting of hydrogen, hydroxyl, and oxo;
R², R³, R⁵, and R⁶ are independently selected from hydrogen, deuterium, and halogen; and
R⁴ is hydrogen or a hydroxyl protecting group.

In some embodiments, W is oxo, R⁴ is a hydroxyl protecting group, and R², R³, R⁵, R⁶ are hydrogen in the compound having a Formula I and the compounds have a Formula I(a):

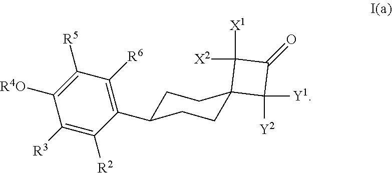

In some embodiments, X¹, X², Y¹, Y², R², R⁴, and R⁶ are hydrogen in the compound having a Formula I and the compounds have a Formula I(b):

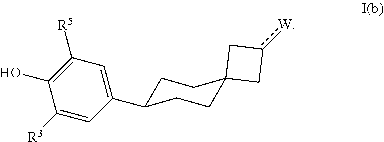

In some embodiments, W and R⁴ are hydrogen in the compound having a Formula I and the compounds have a Formula I(c):

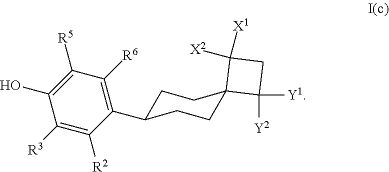

In other embodiments, the disclosed compounds may include an optionally substituted adamantyl group bound to an optionally substituted phenol group (e.g., at a para-position (4-position) of the optionally substituted phenol group) which phenol group optionally is hydroxyl-protected. In some embodiments, the disclosed may have a Formula II:

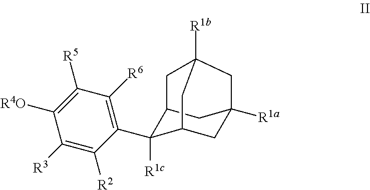

wherein:
R^{1a} and R^{1b} are independently selected from hydrogen, hydroxyl, carboxy alkyl ester, and hydroxy alkyl, optionally with the proviso that R^{1a} and R^{1b} are not the same;
R^{1c} is selected from hydrogen and hydroxyl;
R², R³, R⁵, and R⁶ are independently selected from hydrogen, deuterium, and halogen; and
R⁴ is hydrogen or a hydroxyl protecting group.

In some embodiments, R^{1c} is hydroxyl, R⁴ is a hydroxyl protecting group, and R², R³, R⁵, and R⁶ are hydrogen in the compound having a Formula II and the compounds have a Formula II(a):

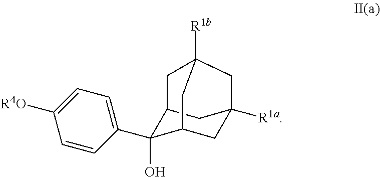

In some embodiments, R^{1c} is hydrogen, R⁴ is a hydroxyl protecting group, and R², R³, R⁵, and R⁶ are hydrogen in the compound having a Formula II and the compounds have a Formula II(b):

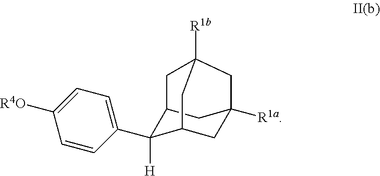

In some embodiments, R^{1c}, R², R³, R⁴, R⁵, and R⁶ are hydrogen in the compound having a Formula II and the compounds have a Formula II(c):

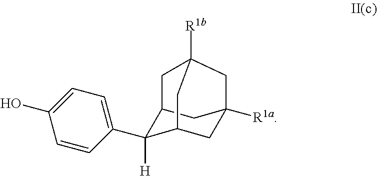

In other embodiments, the disclosed compounds may include an optionally substituted cyclohexyl group bound to an optionally substituted phenol group (e.g., at a para-position (4-position) of the optionally substituted phenol group) which phenol group optionally is hydroxyl-protected. In some embodiments, the disclosed compounds may have a Formula III:

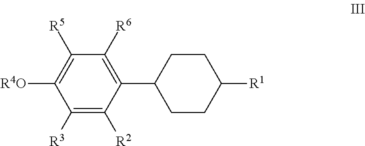

wherein R¹ is selected from hydrogen, hydroxyl, alkyl, hydroxyalkyl, and haloalkyl;
R², R³, R⁵, and R⁶ are independently selected from hydrogen, deuterium, and halogen, with the proviso that if R³, R⁵, and R⁶ are hydrogen, then R¹ is haloalkyl; and
R⁴ is hydrogen or hydroxyl protecting group.

In some embodiments, R², R⁴, and R⁶ are hydrogen, and R¹ is selected from hydroxyalkyl, haloalkyl, and hydroxyl in the compound having a Formula III and the compounds have a Formula III(a):

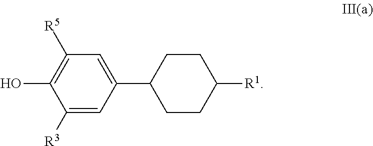

In some embodiments, R², R⁴, and R⁶ are hydrogen, and R¹ is hydroxyalkyl in the compound having a Formula III and the compounds have a Formula III(b):

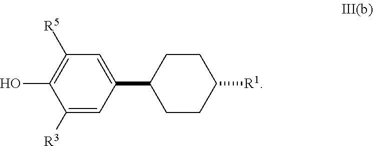

In other embodiments, the disclosed compounds may include an optionally substituted bridged or fused ring system bound to an optionally substituted phenol group (e.g., at a para-position (4-position) of the optionally substituted phenol group) which phenol group optionally is hydroxyl-protected. In some embodiments, the disclosed compounds may have a Formula IV:

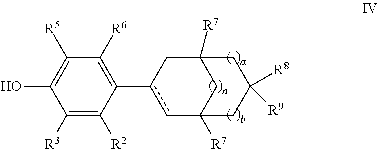

wherein R², R³, R⁵, and R⁶ are independently selected from hydrogen, deuterium, and halogen;
R⁷ is hydrogen or alkyl;
R⁸ and R⁹ are independently selected from the group consisting of hydrogen, hydroxyl, and hydroxyalkyl;
a is 0 or 1;
b is 0 or 1; and
n is 0 or 1.

In some embodiments, n is 0, a and b are 1, and R⁷ is hydrogen or methyl in the compound having a Formula IV and the compounds have a Formula IV(a):

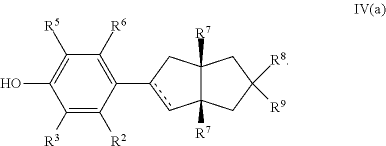

In some embodiments, a, b, and n are 1, and R⁷ is hydrogen in the compound having a Formula IV and the compounds have a Formula IV(b):

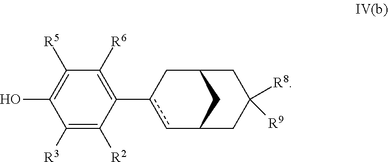

In some embodiments, a, b, and n are 0, and R⁷ and R⁸ are hydrogen, and R⁹ is hydroxymethyl in the compound having a Formula IV and the compounds have a Formula IV(c):

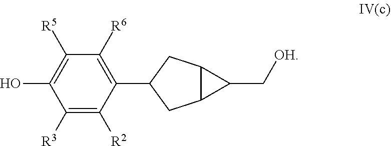

The disclosed compounds may be used to prepare and formulate pharmaceutical compositions. As such, also disclosed herein are pharmaceutical compositions comprising an effective amount of any of the compounds disclosed herein, or pharmaceutically acceptable salts of any of the compounds disclosed herein, together with a pharmaceutically acceptable excipient, carrier, or diluent.

In some embodiments, the disclosed compounds may be used for preparing a medicament for treating a disease or disorder associated with estrogen receptor β (ERβ) activity, and in particular, a disease or disorder that may be treated with an agonist of ERβ. As such, the disclosed compounds may exhibit ERβ agonist activity, and preferably the compounds exhibit specificity as ERβ agonists versus activity as ERβ antagonists and/or versus activity as estrogen receptor α (ERα) agonists and/or activity as ERα antagonists.

### BRIEF DESCRIPTION OF THE DRAWINGS

FIG. 1. Achiral and optically active estrogen receptor-β selective ligands.

FIG. 2. ORTEP of 7-(4-hydroxyphenyl)spiro[3.5]-nonan-2-ol (±)-11.

FIG. 3. Induced-fit docking poses in human ERβ ligand pocket for: (a) 5b (Glide score—10.676) or (b) (S)-11 (Glide score—10.001). Hydrogen bond interactions with the phenol hydroxyl to Glu305 and Arg346, and aliphatic hydroxyl to His475 are indicated by yellow dashed lines. The π-π interaction (dashed blue lines) with Phe356 and Van der Waals interactions with Leu298 is maintained by both ligands.

FIG. 4. CYP450 Enzyme Inhibition Assays. Inhibition of CYP2C9 by 5a/b (red squares, IC₅₀ of 10±0.5 μM) and (±)-11 (blue circles). No significant inhibition of CYP2D6 or CYP3A4 was observed up to 62.5±0.5 μM.

FIG. 5. Predicted CYP450 metabolism. Intrinsic reactivity towards CYP450 hydroxylation of carbon atoms in (a) 5b and (b) 11. Schematic representation of all data for CYP2C9 metabolic predictions (c) for 5b and (d) for 11. The green rays are related to Fe accessibility in docked complexes. Larger and darker circles indicate a higher predicted site of metabolism (SOM) score, proximity to CYP450 heme iron, and higher intrinsic reactivity score. (e) Docking pose for 5b in CYP2C9, showing the phenolic hydrogens closest to the heme Fe.

### DETAILED DESCRIPTION

The present invention is described herein using several definitions, as set forth below and throughout the application.

Unless otherwise specified or indicated by context, the terms “a”, “an”, and “the” mean “one or more.” For example, “a substitution” should be interpreted to mean “one or more substitutions.” Similarly, “a substituent group” should be interpreted to mean “one or more substituent groups.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Disclosed are (4-hydroxylphenyl) substituted carbocycles and their use as selective agonists of the estrogen receptor beta isoform (ERβ). Preferred embodiments of the disclosed compounds include (4-hydroxylphenyl) substituted spiro[5.3]nonanes, (4-hydroxylphenyl) substituted admantanes, (4-hydroxylphenyl) substituted cyclohexanes, (4-hydroxylphenyl) substituted bicyclo[3.3.0]octanes, (4-hydroxylphenyl) substituted bicyclo[3.3.0]octenes, (4-hydroxylphenyl) substituted bicyclo[3.3.1]nonanes, (4-hydroxylphenyl) substituted bicyclo[3.3.1]nonenes, and (4-hydroxylphenyl) substituted bicyclo[3.1.0]hexanes. The disclosed compounds may alternatively be referred to as (4-hydroxyphenyl) substituted carbocycles that include one or more substitutions on the carbocycle substituent, which preferably is a spiro[5.3]nonane substituent, an adamantyl substituent, a cyclohexyl substituent, a bicyclo[3.3.0]octanyl substituent, a bicyclo[3.3.0]octenyl substituent, a bicyclo[3.3.1]nonanyl substituent, a bicyclo[3.3.1]nonenyl substituent, or a bicyclo[3.1.0]hexanyl substituent.

The disclosed compounds may include an optionally substituted spirocyclic substituent, which may include a spiro[5.3]nonane substituent. In some embodiments, the disclosed compounds have a Formula I:

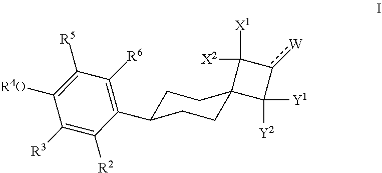

wherein X¹, X², Y¹, and Y² are independently selected from the group consisting of hydrogen, halogen, and hydroxyl;
optionally with the proviso that when X¹ and X² are halogen then Y¹ and Y² are hydrogen and optionally with the proviso that when Y¹ and Y² are halogen then X¹ and X² are hydrogen;
W is selected from the group consisting of hydrogen, hydroxyl, and oxo;
R², R³, R⁵, and R⁶ are independently selected from hydrogen, deuterium, and halogen; and
R⁴ is hydrogen or a hydroxyl protecting group.

In some embodiments, the halogen in the compound of Formula I is chloro.

In some embodiments, the hydroxyl protecting group in the compound of Formula I is a tert-butyldimethylsilyl group.

In some embodiments, W is oxo, R⁴ is a hydroxyl protecting group, and R², R³, R⁵, R⁶ are hydrogen in compounds of Formula I and the compounds have a Formula I(a), where X and Y are as defined for Formula I:

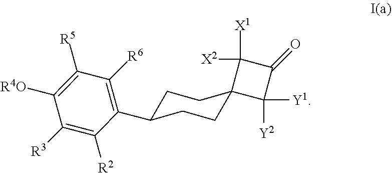

In some embodiments, X¹ and X² are chloro and Y¹ and Y² are hydrogen in compounds of Formula I(a).

In some embodiments, Y¹ and Y² are chloro and X¹ and X² are hydrogen in compounds of Formula I(a).

In some embodiments, X¹, X², Y¹, and Y² are hydrogen in compounds of Formula I(a).

In some embodiments, X¹, X², Y¹, Y², R², R⁴, and R⁶ are hydrogen in compounds of Formula I and the compounds have a Formula I(b), where W, R³, and R⁵ are as defined for Formula I:

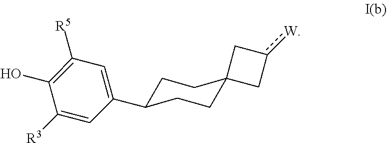

In some embodiments, R³ and R⁵ are hydrogen, and W is oxo in compounds of Formula I(b).

In some embodiments, R³ and R⁵ are hydrogen, and W is hydroxyl in compounds of Formula I(b).

In some embodiments, R³ and R⁵ are deuterium, and W is hydroxyl in compounds of Formula I(b).

In some embodiments, W and R⁴ are hydrogen in compounds of Formula I and the compounds have a Formula I(c), wherein R², R³, RV, R⁶, X¹, X², Y¹, and Y² are as defined for Formula I:

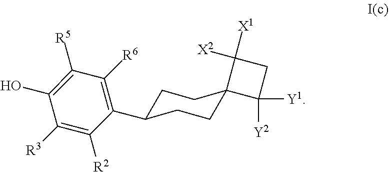

In some embodiments, X¹, X², and Y¹ are hydrogen, and Y² is hydroxyl in compounds of Formula I(c).

In some embodiments, Y¹, Y², and X¹ are hydrogen, and X² is hydroxyl in compounds of Formula I(c).

The disclosed compounds may include an optionally substituted adamantyl substituent. In some embodiments, the compounds have a Formula II:

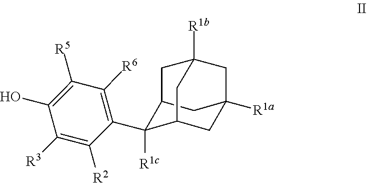

wherein:
R^{1a} and R^{1b} are independently selected from hydrogen, hydroxyl, carboxy alkyl ester, and hydroxy alkyl, optionally with the proviso that R^{1a} and R^{1b} are not the same;
R^{1c} is selected from hydrogen and hydroxyl;
R², R³, R⁵, and R⁶ are independently selected from hydrogen, deuterium, and halogen; and
R⁴ is hydrogen or a hydroxyl protecting group.

In some embodiments, the carboxy alkyl ester in the disclosed compounds of Formula II is carboxy methyl ester (—C(O)OCH₃).

In some embodiments, the hydroxyalkyl in the disclosed compounds of Formula II is hydroxymethyl (—CH₂OH).

In some embodiments, the hydroxyl protecting group in the disclosed compounds of Formula II is a benzyl group (—CH₂-Ph).

In some embodiments, R^{1c} is hydroxyl, R⁴ is a hydroxyl protecting group, R², R³, R⁵, and R⁶ are hydrogen in the disclosed compounds having Formula II and the compounds have a Formula II(a), where R^{1a} and R^{1b} are as defined for Formula II:

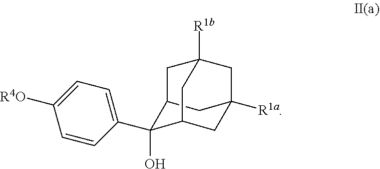

In some embodiments, R^{1b} is carboxy methyl ester (—C(O)OCH₃) and R^{1a} is hydrogen in the compounds of Formula II(a).

In some embodiments, R^{1a} is carboxy methyl ester (—C(O)OCH₃) and R^{1b} is hydrogen in the compounds of Formula II(a).

In some embodiments, R^{1b} is hydroxymethyl (—CH₂OH) and R^{1a} is hydrogen in the compounds of Formula II(a).

In some embodiments, R^{1a} is hydroxymethyl (—CH₂OH) and R^{1b} is hydrogen in the compounds of Formula II(a).

In some embodiments, R^{1c} is hydrogen and R⁴ is a hydroxyl protecting group, and R², R³, R⁵, and R⁶ are hydrogen in the disclosed compounds having Formula II and the compounds have a Formula II(b), where R^{1a} and R^{1b} are as defined for Formula II:

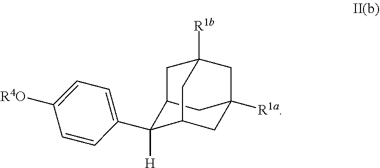

In some embodiments, R^{1b} is hydroxymethyl (—CH₂OH) and R^{1a} is hydrogen in the compounds of Formula II(b).

In some embodiments, R^{1a} is hydroxymethyl (—CH₂OH) and R^{1b} is hydrogen in the compounds of Formula II(b).

In some embodiments, R^{1c}, R², R³, R⁴, R⁵, and R⁶ are hydrogen in the disclosed compounds having Formula II and the compounds have a Formula II(c), where R^{1a} and R^{1b} are as defined for Formula II:

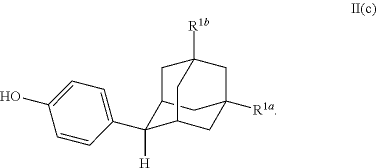

In some embodiments, R^{1b} is hydroxymethyl (—CH₂OH) and R^{1a} is hydrogen in the compounds of Formula II(c).

In some embodiments, R^{1a} is hydroxymethyl (—CH₂OH) and R^{1b} is hydrogen in the compounds of Formula II(c).

The disclosed compounds may include an optionally substituted cyclohexyl substituent. In some embodiments, the compounds have a Formula III:

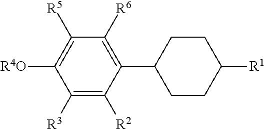

wherein R¹ is selected from hydrogen, hydroxyl, alkyl, hydroxyalkyl, and haloalkyl;
R², R³, R⁵, and R⁶ are independently selected from hydrogen, deuterium, and halogen, with the proviso that if R³, R⁵, and R⁶ are hydrogen, then R¹ is haloalkyl; and
R⁴ is hydrogen or hydroxyl protecting group.

In some embodiments, R², R⁴, and R⁶ are hydrogen, and R¹ is selected from hydroxyalkyl, haloalkyl, and hydroxyl in the disclosed compounds having Formula III.

In some embodiments, R², R⁴, and R⁶ are hydrogen, and R¹ is selected from hydroxyalkyl, haloalkyl, and hydroxyl in the disclosed compounds having Formula III and the compound have a Formula III(a), where R³ and R⁵ are as defined for Formula III:

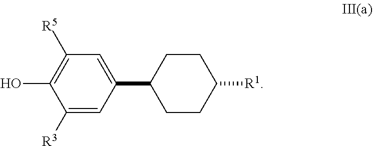

In some embodiments, R¹ is monofluoromethyl (—CH₂F) and R³ and R⁵ are hydrogen in the compounds of Formula III(a).

In some embodiments, R¹ is trifluoromethyl (—CF₃) and R³ and R⁵ are hydrogen in the compounds of Formula III(a).

In some embodiments, R¹ is hydroxyl, R³ is fluoro, and R⁵ is hydrogen in the compounds of Formula III(a).

In some embodiments, R¹ is hydroxymethyl, and R³ and R⁵ are deuterium in the compounds of Formula III(a).

In some embodiments, R², R⁴, and R⁶ are hydrogen, and R¹ is hydroxyalkyl in the disclosed compounds having Formula III and the compounds have a Formula III(b), where R³ and R⁵ are as defined for Formula III:

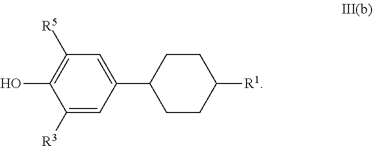

In some embodiments, R³ is fluoro, R⁵ is hydrogen, and R¹ is hydroxymethyl in the compounds of Formula III(b).

In some embodiments, R³ and R⁵ are fluoro, and R¹ is hydroxymethyl in the compounds of Formula III(b).

The disclosed compounds may include an optionally substituted fused or bridged ring system. In some embodiments, the compounds have a Formula IV:

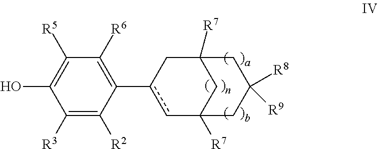

wherein R², R³, R⁵, and R⁶ are independently selected from hydrogen, deuterium, and halogen;
R⁷ is hydrogen or alkyl;
R⁸ and R⁹ are independently selected from the group consisting of hydrogen, hydroxyl, and hydroxyalkyl;
a is 0 or 1;
b is 0 or 1; and
n is 0 or 1.

In some embodiments, n is 0, a and b are 1, and R⁷ is hydrogen or methyl in the disclosed compounds having Formula IV and the compounds have a Formula IV(a), where R², R³, R⁵, R⁶, R⁷, R⁸, and R⁹ are as defined for Formula IV:

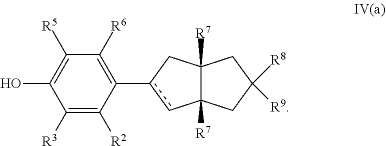

In some embodiments, R⁸ is hydroxyl or hydroxymethyl, and R⁹ is hydrogen in the compounds of Formula IV(a).

In some embodiments, the compounds of Formula IV(a) have a structure:

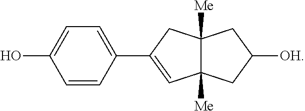 [SMILES: C[C@]12CC(c3ccc(O)cc3)=C[C@@]1(C)CC(O)C2]

In some embodiments, a, b, and n are 1, and R⁷ is hydrogen in the disclosed compounds having Formula IV and the compounds have a Formula IV(b), where R², R³, R⁵, R⁶, R⁸, and R⁹ are as defined for Formula IV:

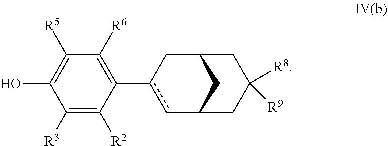

In some embodiments, R⁸ is hydroxyl or hydroxymethyl, and R⁹ is hydrogen in the compounds of Formula IV(b).

In some embodiments, a, b, and n are 0, and R⁷ and R⁸ are hydrogen, and R⁹ is hydroxymethyl in the disclosed compounds having Formula IV and the compounds have a Formula IV(c), where R², R³, R⁵, and R⁶ are as defined for Formula IV:

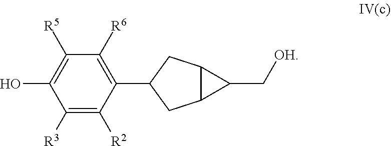

The compounds disclosed herein (e.g., compounds having any of Formula I, I(a), I(b), I(c), II, II(a), II(b), II(c), III, III(a), III(b), IV, IV(a), IV(b), or IV(c)) may have several chiral centers, and stereoisomers, epimers, and enantiomers of the disclosed compounds are contemplated. The compounds may be optically pure with respect to one or more chiral centers (e.g., some or all of the chiral centers may be completely in the S configuration; and/or some or all of the chiral centers may be completely in the R configuration; etc.). Additionally or alternatively, one or more of the chiral centers may be present as a mixture of configurations (e.g., a racemic or another mixture of the R configuration and the S configuration). Compositions comprising substantially purified stereoisomers, epimers, or enantiomers of compound having any of Formula I or II are contemplated herein (e.g., a composition comprising at least about 90%, 95%, or 99% pure stereoisomer, epimer, or enantiomer.

Also disclosed herein are hydroxy-protected derivatives of the compounds disclosed herein. For example, the compounds disclosed herein (e.g., compounds having any of Formula I, I(a), I(b), I(c), II, II(a), II(b), II(c), III, III(a), III(b), IV, IV(a), IV(b), or IV(c)), may include a hydroxy-protected group at any hydroxy group. As contemplated herein, a “protected-hydroxy” group is a hydroxy group derivatized or protected by any of the groups commonly used for the temporary or permanent protection of hydroxy functions (e.g., alkoxycarbonyl, acyl, silyl, or alkoxyalkyl groups). A “hydroxy-protecting group” signifies any group commonly used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl.

As contemplated herein, the word “alkyl” as used in the description or the claims, denotes a straight-chain or branched alkyl radical of 1 to 6 carbons, in all its isomeric forms.

“Alkoxy” refers to any alkyl radical which is attached by oxygen (i.e., a group represented by “alkyl-O—”). Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals.

The term “aryl” specifies a phenyl-, or an alkyl-, nitro- or halogen-substituted phenyl group.

The terms “hydroxyalkyl”, “deuteroalkyl” and “fluoroalkyl” refer to an alkyl radical substituted by one or more hydroxy, deuterium, or fluoro groups respectively.

An “alkylidene” refers to a radical having the general formula CₖH₂ₖ—where K is an integer (e.g., 1-6).

The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halogen, nitro or alkyl substituted benzoyl group.

The term “carboxy alkyl ester” are —C(O)O-alkyl groupings such as carboxy methyl ester, carboxy ethyl ester, carboxy propyl ester, etc. The term “halogen” refers to fluoro, chloro, bromo, and iodo.

As used herein, the symbol “ [symbol] ” in the compounds described herein represents a single bond or a double bond.

The compounds disclosed herein may exhibit binding and agonist and/or antagonist activity for estrogen receptors. As used herein, “ERα” refers to estrogen receptor-alpha, and in particular, human estrogen receptor-alpha. As used herein, “ERβ” refers to estrogen receptor-beta, and in particular human estrogen receptor-beta. Agonists and antagonists for ERα and ERβ are known in the art as are assays for determining the binding affinity of a compound for ERα and ERβ and determining whether a bound compound is an agonist or antagonist for ERα and ERβ. (See e.g., McCullough et al., “Probing the human estrogen receptor-α binding requirements for phenolic mono- and di-hydroxyl compounds: a combined synthesis, binding and docking study,” Biorg. & Med. Chem. (2014) January 1; 22(1):303-10. doi: 10.1016/j.bmc.2013.11.024. Epub (2013) November 21, and the corresponding Supplementary Information, the contents of which are incorporated herein by reference in their entireties). Suitable assays for determining the binding affinity of a compound for ERα and ERβ and determining whether a bound compound is an agonist or antagonist for ERα and ERβ may include fluorescence polarization displacement assays and cell-based ERα and ERβ luminescence activity assays.

As used herein, the term “selective agonist” may be used to refer to compounds that selectively bind to an estrogen receptor, and in particular, ERβ, relative to another estrogen receptor, and in particular ERα. For example, a compound that is a selective agonist for ERβ may have a binding affinity for ERβ receptor (e.g., as measured by K_{d} (nM)) that is at least 3-fold greater (or at least 5-fold greater, at least 10-fold greater, at least 20-fold greater, at least 50-fold greater, at least 100-fold greater, at least 500-fold greater, or at least 1000-fold greater) than a binding affinity for ERα. Preferably, a selective agonist for ERβ has a K_{d} (nM) for ERβ that is less than 100 nM, more preferably less than 10 nM, or even more preferably less than 1 nM; and preferably, a selective agonist for ERβ has a K_{d} (nM) for ERα that is greater than 500 nM, more preferably greater than 1000 nM, or even more preferably greater than 2000 nM.

As used herein, the term “selective agonist” may be used to refer to compounds that selectively bind and agonize an estrogen receptor, and in particular ERβ, relative to another estrogen receptor, and in particular ERα. For example, a compound that is a selective agonist for ERβ may have an IC₅₀ (nM) in an assay for ERβ receptor agonist activity that is less than 100 nM, preferably less than 10 nM, even more preferably less than 1 nM; and a compound that is that is a selective agonist for ERβ may have an IC₅₀ (nM) in an assay for ERα receptor agonist activity that is greater than 100 nM, preferably greater than 500 nM, even more preferably greater than 1000 nM.

As used herein, the term “selective agonist” may be used to refer to compounds that selectively bind and agonize an estrogen receptor, and in particular ERβ, instead of antagonizing an estrogen receptor, and in particular ERβ. For example, a compound that is a selective agonist for ERβ may have an IC₅₀ (nM) in an assay for ERβ receptor agonist activity that is less than 100 nM, preferably less than 10 nM, even more preferably less than 1 nM; and a compound that is that is a selective agonist for ERβ may have an IC₅₀ (nM) in an assay for ERβ receptor antagonist activity that is greater than 100 nM, preferably greater than 500 nM, even more preferably greater than 1000 nM.

Pharmaceutically acceptable salts of the disclosed compounds also are contemplated herein and may be utilized in the disclosed treatment methods. For example, a substituent group of the disclosed compounds may be protonated or deprotonated and may be present together with an anion or cation, respectively, as a pharmaceutically acceptable salt of the compound. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

It should be recognized that the particular counter-ion forming a part of any salt of a compound disclosed herein is usually not of a critical nature, so long as the salt as a whole is pharmacologically acceptable and as long as the counterion does not contribute undesired qualities to the salt as a whole. Undesired qualities may include undesirably solubility or toxicity.

It will be further appreciated that the disclosed compounds can be in equilibrium with various inner salts. For example, inner salts include salts wherein the compound includes a deprotonated substituent group and a protonated substituent group.

The disclosed compounds may be used to prepare and formulate pharmaceutical compositions. As such, also disclosed herein are pharmaceutical compositions comprising an effective amount of any of the compounds disclosed herein, or pharmaceutically acceptable salts of any of the compounds disclosed herein, together with a pharmaceutical excipient. In some embodiments, the disclosed compounds may be used for preparing a medicament for treating a disease or disorder associated with estrogen receptor β (ERβ) activity, and in particular, a disease or disorder that may be treated with a specific agonist of ERβ. As such, the disclosed compounds may exhibit ERβ agonist activity, and preferable the compounds exhibit specificity as an ERβ agonist versus an ERβ antagonist, an ERα agonist, and/or an ERα antagonist.

The disclosed compounds may be used to prepare and formulate pharmaceutical compositions for treating diseases that are associated with estrogen ERβ activity. Diseases and disorders associated with ERβ activity may include, but are not limited to, cell proliferative diseases and disorders (e.g., breast cancer, ovarian cancer, and endometrial cancer), psychiatric diseases and disorders (e.g., depression or anxiety), vasomotor diseases and disorders (e.g. hot flashes), neurodegenerative diseases or disorders, bone metabolic diseases or disorders (e.g. osteoporosis), metabolic diseases or disorders (e.g., obesity or insulin resistance), and cardiovascular diseases or disorders. The disclosed pharmaceutical compositions may be administered to patients in need thereof in methods for treating diseases and disorders associated with ERβ activity.

The compounds and pharmaceutical compositions disclosed herein may be administered to a patient in need thereof to treat a disease or disorder. In some embodiments, the compounds disclosed herein may be administered at an effective concentration such that the compound functions as an agonist for ERβ in order to treat a disease or disorder associated with ERβ activity. In some embodiments, the amount of the disclosed compounds that is effective for the compound to function as an agonist of ERβ is about 0.05-50 μM (or about 0.05-10 μM, or about 0.05-1 μM).

As used herein, a “patient” may be interchangeable with “subject” or “individual” and means an animal, which may be a human or non-human animal, in need of treatment. Suitable patients for the disclosed methods may include, for example mammals, such as humans, monkeys, dogs, cats, horses, rats, and mice. Suitable human patient include, for example, those who have a disease or disorder associated with ERβ activity or those who have been determined to be at risk for developing a disease or disorder associated with ERβ activity.

As used herein, a “patient in need of treatment” may include a patient having a disease, disorder, or condition that is responsive to therapy with an ERβ agonist. For example, a “patient in need of treatment” may include a patient having a cell proliferative disease, disorder, or condition such as cancer (e.g., cancers such as breast cancer). In addition, a “patient in need of treatment” may include a patient having a psychiatric disease or disorder (e.g., depression or anxiety). Moreover, a “patient in need of treatment” may include a patient having a vasomotor disease or disorder (e.g., hot flashes).

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with ERβ activity in a patient, whereby the effective amount induces, promotes, or causes ERβ agonist activity in the patient.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual patient; the particular compound administered; the mode of administration; the bioavailability characteristics of the preparation administered; the dose regimen selected; the use of concomitant medication; and other relevant circumstances.

In some embodiments, a daily dose of the disclosed compounds may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. The dose may be administered under any suitable regimen (e.g., weekly, daily, twice daily).

The pharmaceutical compositions for use according to the methods as disclosed herein may include be a single compound as an active ingredient or a combination of compounds as active ingredients. For example, the methods disclosed herein may be practiced using a composition containing a single compound that is an ERβ agonist. Alternatively, the disclosed methods may be practiced using a composition containing two or more compounds that are ERβ agonists, or a compound that is an ERβ agonist together with a compound that is an ERα antagonist.

Instead of administering a pharmaceutical composition comprising a compound that is an ERβ agonist together with a compound that is an ERα antagonist, the disclosed methods may be practiced by administering a first pharmaceutical composition (e.g., a pharmaceutical composition comprising an ERβ agonist) and administering a second pharmaceutical composition (e.g., a pharmaceutical composition comprising an ERα antagonist), where the first composition may be administered before, concurrently with, or after the second composition. As such, the first pharmaceutical composition and the second pharmaceutical composition may be administered concurrently or in any order, irrespective of their names.

As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the compounds utilized in the methods disclosed herein may be formulated as a pharmaceutical composition in liquid form (e.g., an injectable liquid or gel)

The compounds utilized in the methods disclosed herein may be formulated as a pharmaceutical composition that includes an excipient, carrier, or diluent. For example, the excipient, carrier, or diluent may be selected from the group consisting of proteins, carbohydrates, sugar, talc, magnesium stearate, cellulose, calcium carbonate, and starch-gelatin paste.

The compounds utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and crosslinked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents for the pharmaceutical compositions may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

The disclosed pharmaceutical compositions also may include disintegrants. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

The disclosed pharmaceutical compositions also may include effervescent agents. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### EXAMPLES

The following examples are illustrative and should not be interpreted to limit the claimed subject matter.

Example 1. Discovery of two novel (4-hydroxyphenyl) substituted polycyclic carbocycles as potent and selective estrogen receptor beta agonists. See also Wetzel et al., Discovery of Two Novel (4-Hydroxyphenyl) Substituted Polycyclic Carbocycles as Potent and Selective Estrogen Receptor Beta Agonists, Bioorg. Med. Chem. Lett., 73(2022) 128906, the contents of which are incorporated herein by reference in its entirety.

### Abstract

Two (4-hydroxyphenyl) substituted polycyclic carbocycles were prepared and assayed for estrogen receptor activity. 4-(4-Hydroxyphenyl)tricyclo[3.3.1.1^{3,7}]decane-1-methanol (5a/b) and 7-(4-hydroxyphenyl)spiro[3.5]nonan-2-ol ((±)-11) were found to be potent ERb agonists (1.9±0.4 nM and 6.2±1.4 nM respectively) in a cell-based functional assay. Furthermore, both 5a/b and 11 were highly selective for ERb over ERa (377 and 1,100-fold selective respectively). While neither compound inhibited CYP2D6 or CYP3A4 at concentrations up to 62.5 mM, 5a/b did have weak binding to CYP2C9 with an IC₅₀ of 10±0.5 mM. Computational assessment of 5a/b and 11 predicted the most probable site of metabolism would be ortho to the phenolic hydroxyl group.

### 1. Introduction

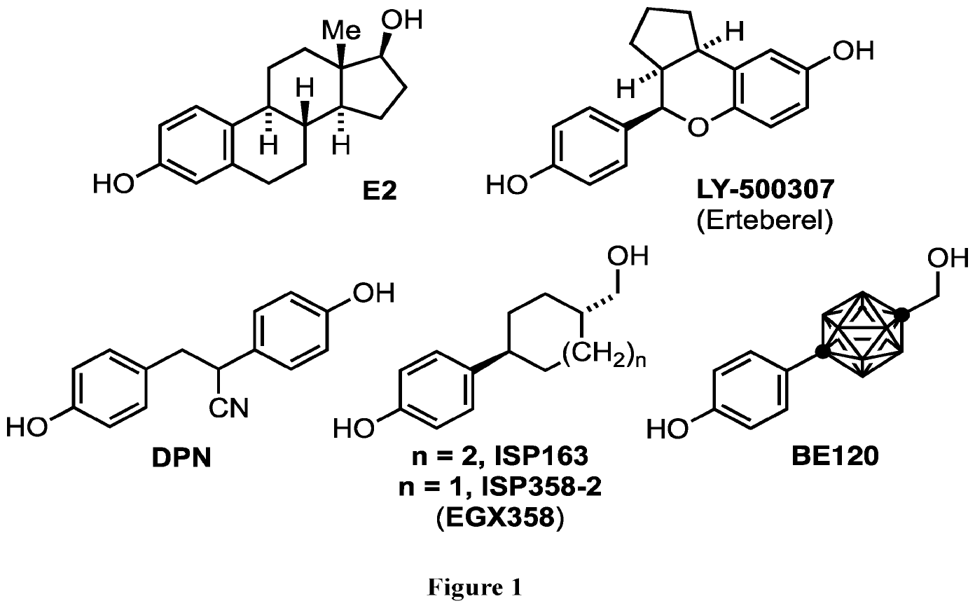

The estrogen receptors α and β (ERα and ERβ) belong to the nuclear hormone family of intracellular receptors for which 17β-estradiol (E2, FIG. 1) is the endogenous ligand. The two receptors exhibit overlapping but distinct patterns of tissue distributions as well as different types of transcriptional regulation.¹ Menopause results in a marked decrease in the production of estrogens and as such is associated with adverse symptoms such as hot flashes and memory decline. Hormone replacement therapy (HRT) consisting of estradiol or conjugated estrogens has been utilized to alleviate these symptoms, as well as address bone density loss,² however HRT is associated with an increased risk of breast cancer and blood clots leading to stroke.³ Activation of ERα, but not ERβ, is responsible for the increased health risks of HRT.⁴

Crystal structures of E2 with human ERα and ERβ reveal hydrogen bonding interactions between the phenolic hydroxyl group with a bound water molecule and two amino acid residues (Glu353 and Arg394 in ERα, Glu305 and Arg346 in ERβ) and the aliphatic hydroxyl with a histidine residue (His524 in ERα, His475 in ERβ).⁵ These interactions are spaced ˜11 Å apart. Aside from these hydrogen bonding interactions, the remainder of the ligand binding pocket consists of a lipophilic cavity into which other agonists can bind.

The search for selective estrogen receptor-beta agonists (SERBAs) has resulted in the discovery of a number of such compounds. Two such non-steroidal SERBAs are LY-500307 (Erteberel, EC₅₀=0.66 nM, 32-fold selective)⁶ and DPN (EC₅₀=66.0 nM, 78-fold selective).⁷ We have recently reported two non-steroidal SERBAs, ISP163 (EC₅₀=33±5 nM, 318-fold selective)⁸ and ISP358-2/EGX358 (EC₅₀=27±4 nM, 750-fold selective).⁹ Long-term oral dosing of EGX358 (0.5 mg/Kg) has shown efficacy for memory consolidation and mitigates drug-induced vasodilation in an ovariectomized mouse model for menopause.¹⁰ The disubstituted 1,12-dicarba-closo-dodecaborane BE120, containing a large lipophilic linker between a phenol and a hydroxymethylene group, is ˜100 fold more potent than E2, but exhibits low selectivity (ERβ:ERα=1.4).^{11a-c} More recently, Bartunek and Coss^{11d} have demonstrated that adding alkyl groups to the BE120 structure (e.g. A, FIG. 1) improves ERβ selectivity (˜200 fold selective) albeit at the expense of potency (EC₅₀˜20-30 nM). Similar to dicarbadodecaborane, the adamantane moiety “is viewed as providing just the critical lipophilicity” for known phamacophores.¹² With inspiration from these features, we herein report the synthesis and evaluation of two new potent and selective ERβ agonists.

### 2. Results and Discussion

### 2.1 Chemistry

Esterification of 2-adamantanone-5-carboxylic acid with methanol/thionyl chloride gave the known¹³ methyl ester 1 (Scheme 1). Addition of a slight excess of 4-benzyloxyphenyl Grignard reagent (1.04 equiv) to 1 gave stereoisomeric tertiary alcohols 2a/b; this was determined to be a ˜1:1 mixture by integration of the methyl ester singlets (δ 3.67 and 3.54 ppm). Reduction of the mixture 2a/b with LiAlH₄ gave a mixture of primary alcohols 3a/b; ionic reduction of this mixture afforded a mixture of 4a/b. Finally, benzylic ether cleavage of 4a/b using H₂ and 10% Pd/C catalyst afforded 5a/b. This was revealed to be a 1.8:1 mixture based on integration of the alcohol methylene singlets (δ 3.18 and 2.99 ppm).

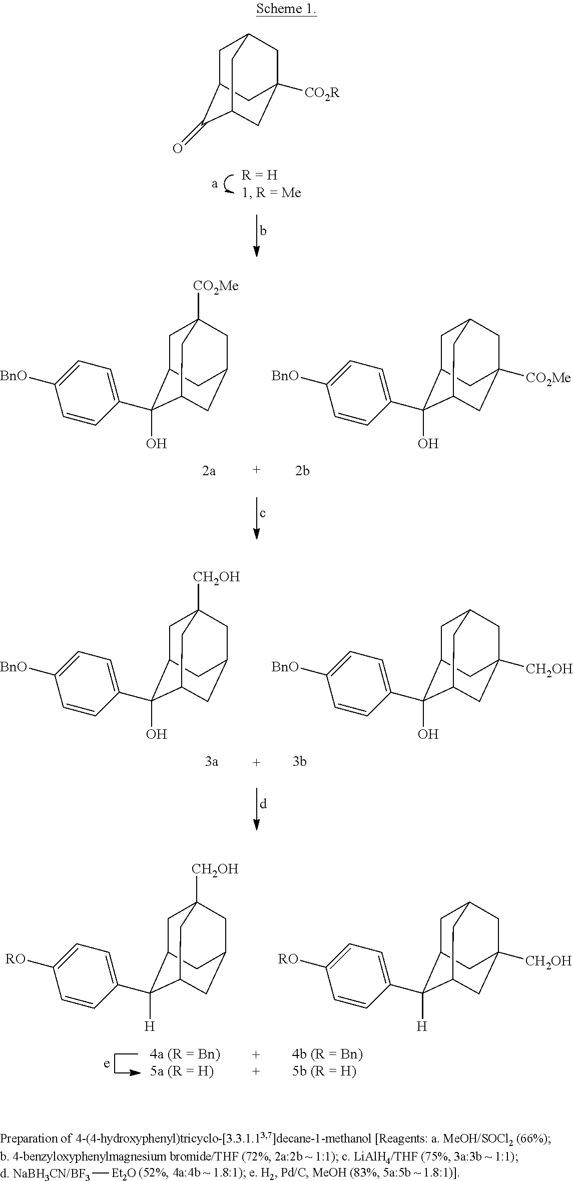

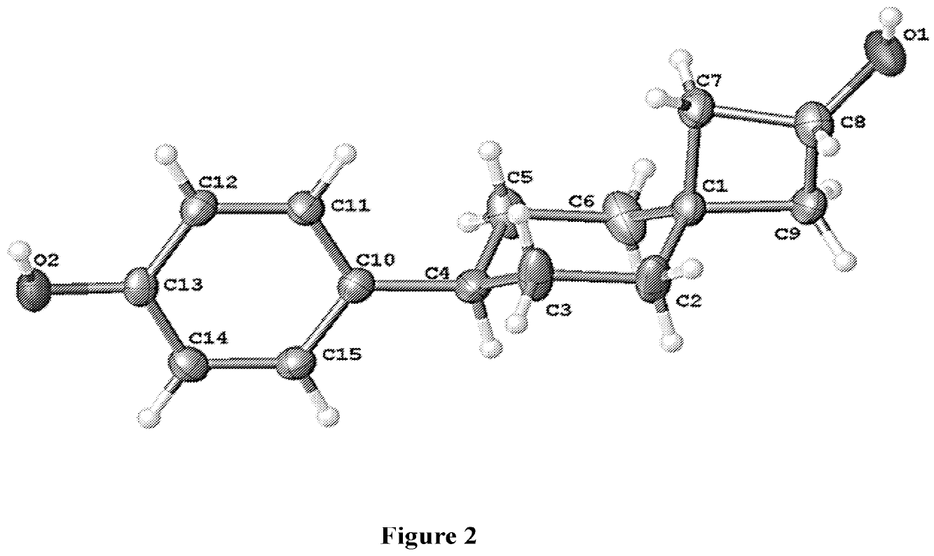

Recognizing that the primary alcohol functionality present in 5a/b represented a potential metabolic liability, we reasoned that an analog containing a cyclobutanol functionality would be less prone to oxidation. Toward this end, 4-[4-(t-butyldimethylsilyl-oxy)phenyl]methylenecyclohexane⁹ (6, Scheme 2) reacted with dichloroketene,¹⁵ generated by the reduction of trichloroacetyl chloride with zinc, led to an inseparable mixture of spirocyclic dichlorocyclobutanones 7 and 8, resulting from exo- and endo-addition. Reduction of the mixture with Zn in acetic acid gave cyclobutanone 9, which was deprotected by reaction with HF-pyridine to afford 10. The structure of 10 was tentatively based on the known regioselectivity for dichloroketene cycloaddition to methylenecyclohexanes,^{16a} as well as its ¹H NMR spectral data. In particular the signals for the cyclobutanone methylene protons of 10 appear as two singlets (S 2.81 and 2.77 ppm). This lack of ³J_{H-H} for each signal is in contrast to that anticipated for a 2,2-disubstituted cyclobutanone in which the methylene protons appear as two triplets (J˜8 Hz).^{16b} Reduction of 10 under Luche conditions gave the cyclobutanol (±)-11. The structure of 11 was tentatively assigned on the basis of its NMR spectral data. In particular, the signal for the 2° alcohol CH proton appears at δ 4.21 as a narrow pentet (J=7.3 Hz). This tentative structural assignment was corroborated by X-ray crystal diffraction (FIG. 2) which indicated an O—O distance of 11.4 Å.¹⁴

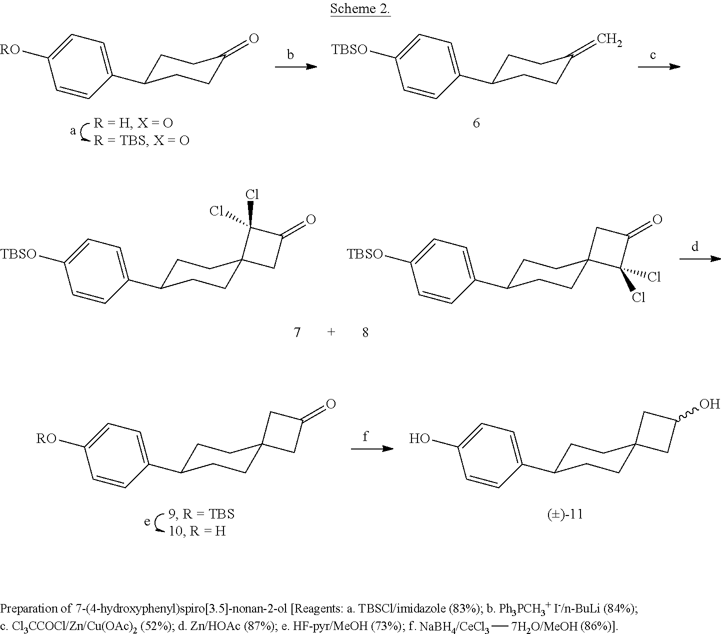

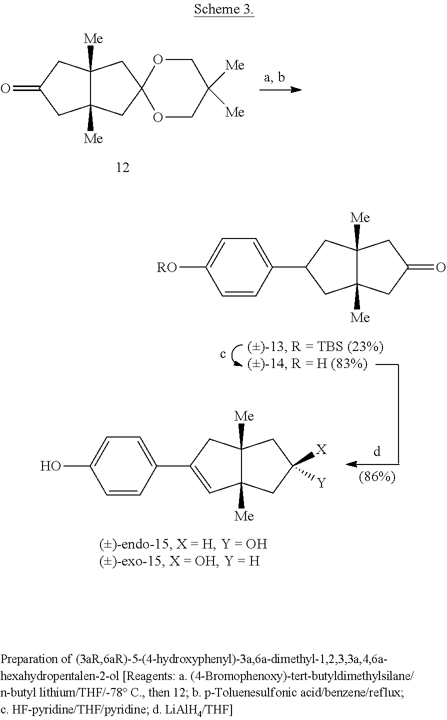

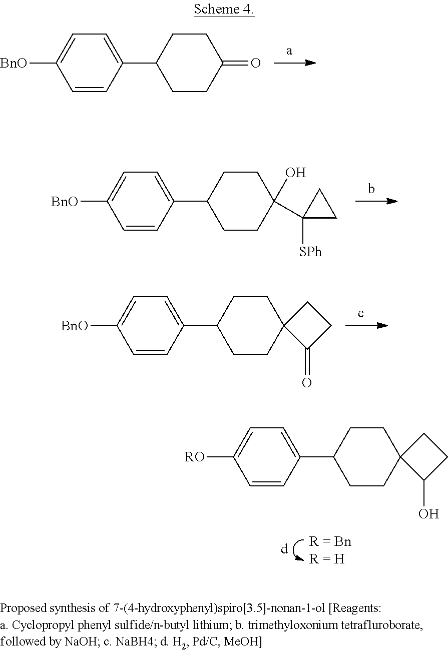

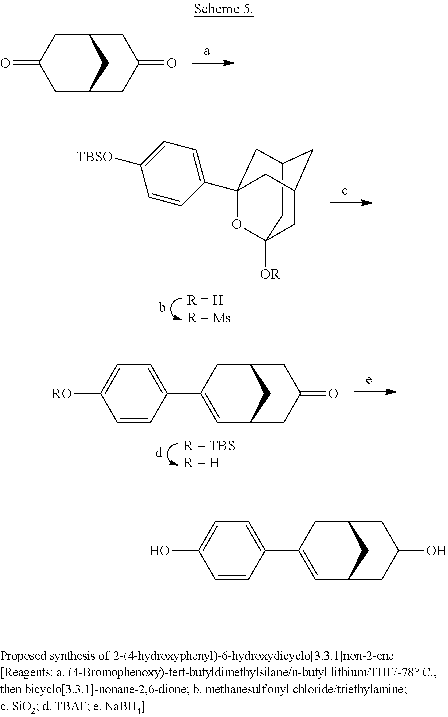

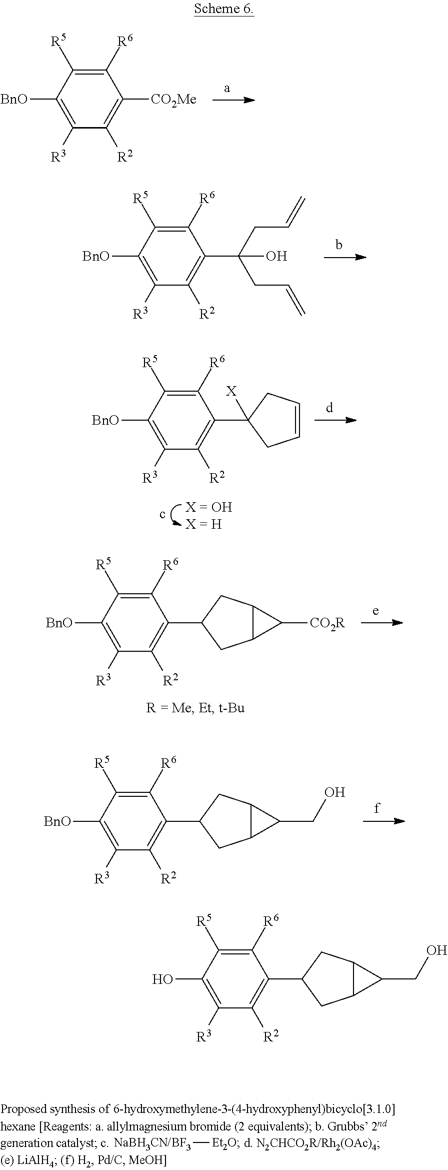

### 2.2 Biological Activity Evaluation

### 2.2.1. Binding and Cell-Based Assays

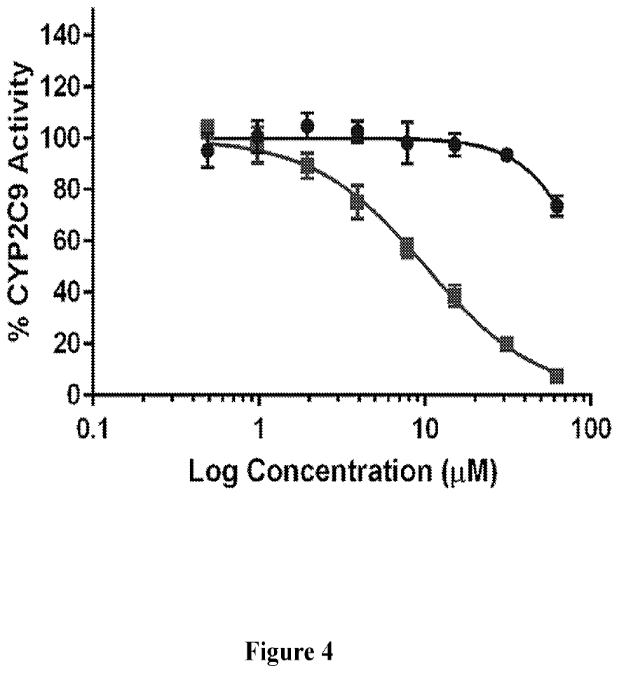

Affinity for 5a/b (IC₅₀=1.3 nM) is 18-fold higher than the previously reported lead molecule, EGX358,⁹ and (±)-11 is 6-fold more potent (Table 1, supplementary FIG. S1). The TR-FRET binding assay measures the ability to displace a fluorescently labeled E₂ agonist from the ligand binding domain, so do not reflect ability to bind in the presence of coactivator or to activate transcription, as agonists. When the co-activator form of the TR-FRET LBD assay was performed 5a/b and 11 were 9.5 and 2.3 fold more potent than EGX358 (Table 1, supplementary FIG. S2) for binding to ER and recruiting the PPARγ coactivator peptide. This assay measures activation of the ER LBD, in that it measures binding and agonist-induced recruitment of the coactivator peptide rather than simply binding of agonist to the receptor. Finally, agonist and antagonist activity of 5a/b and (±)-11 were measured in a cell-based transcriptional activation assay (Table 1, supplementary FIG. S3). In this assay, a full length and native ER is used (in contrast to just the LBD) to best mimic the in vivo situation. Adamantylphenol 5a/b displayed the greatest potency of the three compounds, with an ERβ EC₅₀ of 1.9±0.4 nM. When compared to the ERα activation, 5a/b demonstrated 377-fold selectivity for ERβ (FIG. 4a). Spirocyclic butanol (±)-11 is less potent than 5a/b but more selective, with an ERβ EC₅₀ of 6.2±1.6 nM and 1,100-fold selectivity for ERβ over ERα (FIG. 4b). Both compounds are more potent than EGX358, but 5a/b experiences a loss of selectivity relative to EGX358.

TABLE 1
Biological evaluation in TR-FRET binding and cell-based transcriptional assays. IC₅₀/EC₅₀ values in nM.

| | | | TR-FRET | | | | | |
|---|---|---|---|---|---|---|---|---|
| | TR-FRET | TR-FRET | coactivator | Cell-based transcription assay | | | | |
| | ligand | coactivator | binding | ERβ | ERβ | ERα | ERα | ERβ/ERα |
| | displacement | binding | ERβ:ERα | agonism | antagonism | agonism | antagonism | agonist |
| entry | ERβ IC₅₀ | ERβ IC₅₀ | selectivity | EC₅₀ | EC₅₀ | EC₅₀ | EC₅₀ | selectivity |
| E2 | 0.3 ± 0.2 | 1.9 ± 0.3 | — | 0.022 ± 0.005 | ND | 0.31 ± 0.03 | ND | 14 |
| EGX358^{a} | 24 ± 2 | 191 ± 15 | 15 | 27 ± 4 | >10,000 | 20,400 ± 860 | >10,000 | 750 |
| 5a/b | 1.3 ± 0.6 | 83 ± 12 | 9 | 1.9 ± 0.4 | >10,000 | 717 ± 149 | >10,000 | 377 |
| 10 | 2221 ± 2657 | 1639 ± 301 | ND | ND | ND | ND | ND | — |
| (±)-11 | 4.4 ± 0.6 | 20 ± 3 | 5 | 6.2 ± 1.4 | >10,000 | 6,700 ± 308 | >10,000 | 1,100 |

^{a}data for EGX358 from ref. 8

### 2.2.2 Estrogen Receptor Docking Results

The ERβ vs. ERα selectivities of 5a/b and (±)-11 are considerably greater in the cell-based functional assay than in the TR-FRET coactivator binding assay. The TR-FRET coactivator binding assays for either ERβ or ERα use only the ligand binding domain, and thus this assay only measures binding synergy that is induced by coactivator binding. In comparison, the more biologically relevant cell-based assay measures a dose-response effect based on activation of transcription in the nucleus that results from a conformational change induced by binding of the estrogen receptor to the agonists—from rotation of Helix-12 that permits binding coactivators that are part of the activated transcription initiation complex. In other words, it is measuring actual agonist activity in the cell, a multistep process, rather than just simple affinity for the receptor.

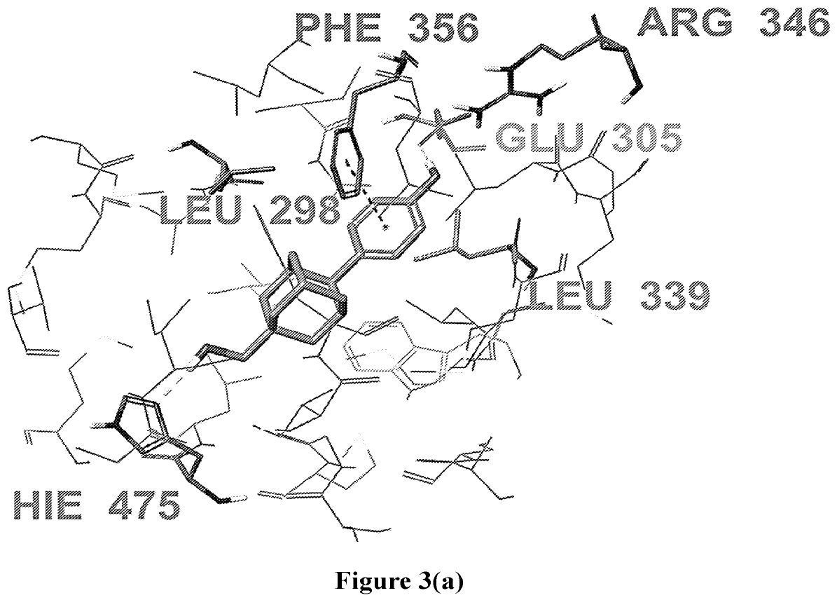

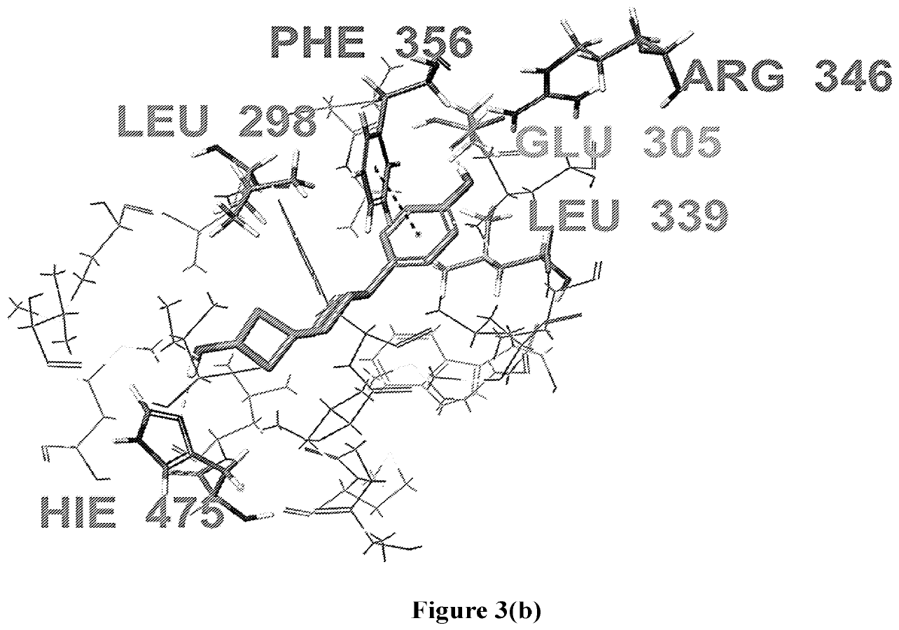

Glide Induced Fit Docking, within the Schrodinger Suites, into the ligand binding pocket of ERβ (PDB ID: 2JJ3) was conducted for adamantyl phenols 5a and 5b and spirocyclobutanol (S)-11 and (R)-11 (FIG. 3, supplemental figures S4a-d). All structures docked with high affinity (−9.885 to −10.823 kcal/mol) in the ERβ ligand pocket, with the phenol hydroxyl group hydrogen bonding with the Glu305-Arg346-water triad, as is typical for estrogens and SERBAs.⁹ At the other end of the pocket, 11 Å away, the aliphatic hydroxyl of 5b is hydrogen bonding to the His475 δ1 nitrogen; this hydrogen bonding is lacking for aliphatic hydroxyl of stereoisomer 5a which may contribute to the lesser docking for this stereoisomer. All have an important π-π interaction with Phe356.

The spirocyclobutyl ring of (S)-11 and (R)-11, appears to position the aliphatic hydroxyl well for hydrogen bonding to His475. Surprisingly, induced fit docking of 5a, 5b, (S)-11 and (R)-11 into the ligand binding pocket of ERα (PDB ID: 1ere) gave docking energies similar to those for docking into ERβ. These similar docking energies are more aligned with the ERβ:ERα selectivity observed in the TR-FRET coactivator binding than they are with the cell-based functional assay. We have previously observed this in docking of EGX358 with ERβ and ERα.⁹

### 2.2.3 CYP450 Metabolism

Experimental measurements of CYP450 binding, in CYP450 enzyme kinetic inhibition assays, indicates that of the three CYP450s tested (CYP3A4, CYP2D6, CYP2C9), there is weak binding only to CYP2C9 by 5a/b with an IC₅₀ of 10±0.5 μM (FIG. 4, supplemental figures S5a-b). In contrast, spirocyclobutanol (±)-11 does not bind to any of these CYP450 enzymes with significant activity making 11 a preferable drug lead molecule in terms of potential metabolic liabilities.

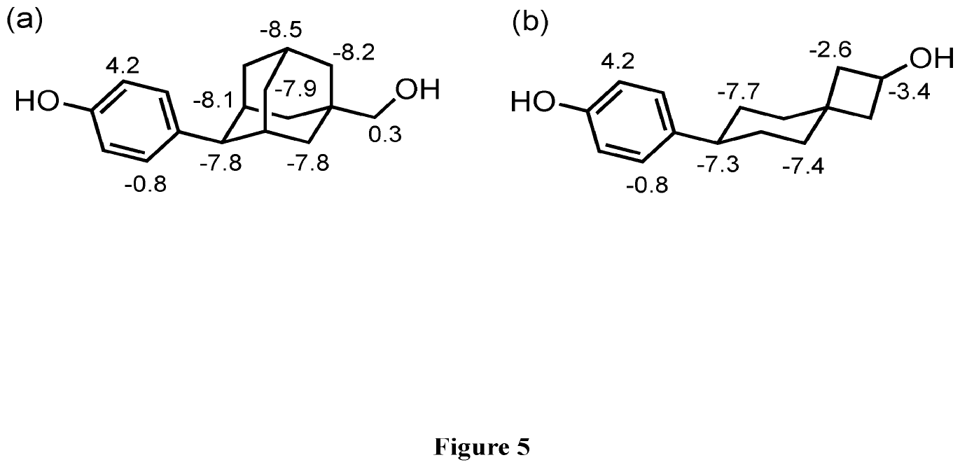

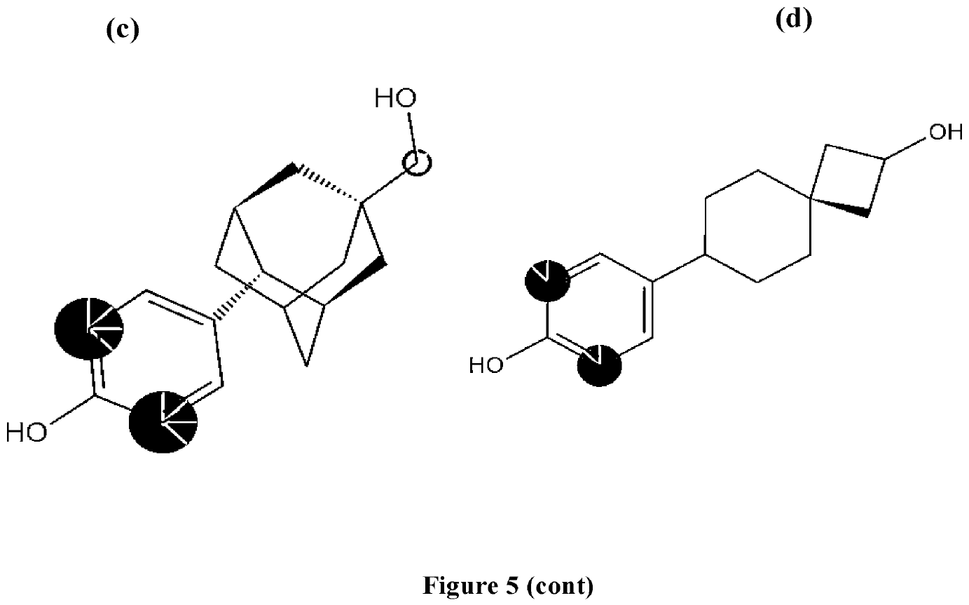

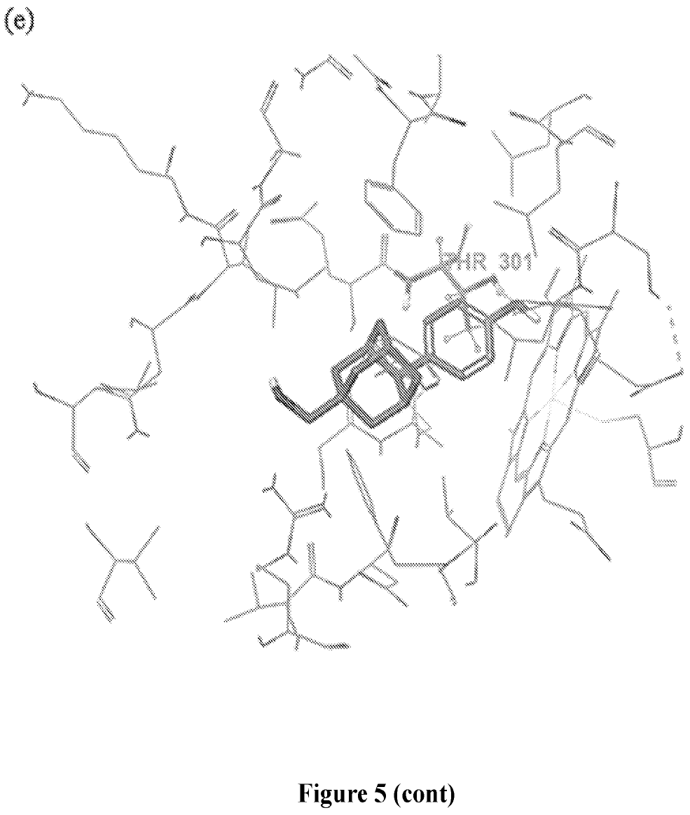

Predicted metabolism of 5b and 11, based on Schrodinger calculations, are shown in FIG. 5, indicating the most likely site of metabolism on both molecules is the phenol ring, especially ortho to the phenolic hydroxyl group (FIGS. 5a-d). This is based both in intrinsic reactivity of the position as well as docking into the CYP450 active site pocket (FIG. 5e). While the aliphatic hydroxyls can potentially be oxidized as well, the primary alcohol of 5b (FIG. 5a) is predicted to be much more labile than the secondary alcohol of 11 (FIG. 5b), as expected.

Bioavailability predictions were made using Schrodinger QikProp. Predicted transport across the intestinal mucosa, as Caco-2 permeability was greatest for 5b (1126 nm/sec), followed by EGX358 (1005 nm/sec) and then 11 (935 nm/sec). Predicted transport across the blood brain barrier (BBB) as MDCK permeability, was greatest for 5b (562 nm/sec), followed by EGX358 (497 nm/sec) and then 11 (460 nm/sec). In all cases though, relatively favorable transport properties are predicted, consistent with our previously reported^{9,10} efficacy studies using orally delivered EGX358 in mouse model studies.

### CONCLUSION

In summary, two new polycyclic 4-substituted phenols 5a/b and (±)-11 were prepared. Each was found to be a single-digit nanomolar ERβ agonist in a cell-based functional assay (EC₅₀=1.9±0.4 and 6.2±1.4 nM respectively). The spirocyclobutanol analog 11 exhibited outstanding ERβ:ERα selectivity (1,100-fold selective). In addition, 11 did not inhibit the P450 enzymes CYP2C9, CYP2D6 or CYP3A4 up to 62.5 μM. While one of the predicted sites for metabolism of 5a/b was the primary alcohol functionality, computational predictions indicated that the cyclobutanol functionality of 11 was much less prone to reactivity. Future studies will involve separation of the stereoisomers 5a/b and of the enantiomers (S)-11 and (R)-11, assessment of their individual activation of ERβ and ERα, in vivo testing for efficacy in moderating hot flashes and consolidating memory in an ovariectomized mouse model, as well as microsomal stability, PK and safety toxicology. These results will be reported in due course.

### 4. Experimental

### 4.1 Chemistry

### 4.1.1 General Experimental

All reactions involving moisture or air sensitive reagents were carried out under a nitrogen atmosphere in oven-dried glassware with anhydrous solvents. THF and ether were distilled from sodium/benzophenone. Purifications by chromatography were carried out using flash silica gel (32-63μ). NMR spectra were recorded on either a Varian Mercury+300 MHz or a Varian UnityInova 400 MHz instrument. CDCl₃, CD₃OD and d₆-DMSO was purchased from Cambridge Isotope Laboratories. ¹H NMR spectra were calibrated to 7.27 ppm for residual CHCl₃ or 3.31 ppm for CD₂HOD. ¹³C NMR spectra were calibrated from the central peak at 77.23 ppm for CDCl₃ or 49.15 ppm for CD₃OD. Coupling constants are reported in Hz. High-resolution mass spectra were obtained from the mass spectroscopy facility at University of Wisconsin-Milwaukee.

### 4.1.2 Methyl 4-oxoadamantane-1-carboxylate (1)

To a solution of 2-adamantanone-5-carboxylic acid (5.00 g, 25.7 mmol) in methanol (50 mL) was added dropwise SOCl₂ (4.67 mL, 64.4 mmol). The solution was heated at reflux for 6 h. The solution was cooled to room temperature and quenched with water (20 mL). Methanol was evaporated under reduced pressure followed by addition of saturated aqueous sodium bicarbonate (10 mL). The resulting mixture was extracted several times with ethyl acetate, the combined organic layers were washed with brine, dried (MgSO₄), and concentrated in vacuo to afford 1 as a colorless solid (3.520 g, 66%). ¹H NMR (400 MHz, CDCl₃) δ 3.60 (s, 3H), 2.67-2.37 (m, 1H), 2.28-1.25 (m, 12H); ¹³C NMR (100 MHz, CDCl₃) δ 216.4, 175.9, 51.7, 45.5, 39.8, 38.0, 37.5, 27.0 ppm. The spectral data for this compound were consistent with the literature spectral data.¹³

### 4-(4-Benzyloxyphenyl)-4-hydroxytricyclo[3.3.1.1^{3,7}]-decane-1-carboxylic Acid Methyl Ester (2a/b)

To a solution of 1 (1.20 g, 5.76 mmol) in dry THF (30 mL) at 0° C. was added dropwise a solution of 4-benzyloxyphenyl-magnesium bromide (1.0 M in THF, 6.0 mL, 6.0 mmol). The reaction was warmed to room temperature and stirred for 4 h. the reaction was quenched with saturated NH₄Cl (20 mL) and partitioned between ether (30 mL) and water (20 mL). The aqueous layer was extracted several times with ether, and the combined ethereal layers were washed with brine, dried (MgSO₄), and concentrated. The residue was purified by column chromatography (SiO₂, hexanes-ethyl acetate=4:1) to give 2a/b (1.620 g, 72%) as a dark yellow paste. NMR spectroscopy revealed this to be a mixture of two stereoisomers (˜1:1 ratio by integration). ¹H NMR (400 MHz, CDCl₃) δ 7.48-7.32 (m, 7H, ArH), 7.01-6.95 (m, 2H, ArH), 5.07 (s, 2H, CH₂Ph), 3.68 (s, 1.5H, OMe), 3.56 (s, 1.5H, OMe), 2.65-2.55 (m, 3H), 2.42-2.37 (m, 1H), 1.90-1.83 (m, 6H), 1.70-1.64 (m, 3H); ¹³C NMR (100 MHz, CDCl₃) δ 177.9, 177.5, 158.0, 157.9, 137.0, 136.9, 128.6, 128.0, 127.5, 126.7, 126.5, 114.9, 114.8, 74.5, 74.3, 69.9, 51.6, 40.3, 39.7, 39.0, 38.9, 36.2, 35.6, 35.3, 34.2, 33.7, 31.8, 27.1, 26.6 ppm. HRMS: M−H⁺. found 391.1920. C₂₅H₂₈O₄—H⁺ requires 391.1915.

### 4-(4-Benzyloxyphenyl)-4-hydroxytricyclo[3.3.1.1^{3,7}]-decane-1-methanol (3a/b)

To a solution of 2a/b (1.50 g, 4.96 mmol) in dry THF (20 mL) at 0° C. was added solid LiAlH₄ (753 mg, 19.8 mmol). The reaction was warmed to room temperature and stirred for 2 h. The reaction was cautiously quenched with water (15 mL) and extracted several times with ethyl acetate. The combined organic layers were dried (MgSO₄) and concentrated. The residue was purified by column chromatography (SiO₂, hexanes-ethyl acetate=7:3) to give 3a/b (1.330 g, 74%) as a colorless solid. NMR spectroscopy revealed this to be a mixture of two stereoisomers (1:1 ratio by integration). ¹H NMR (400 MHz, CD₃OD) δ 7.48-7.27 (m, 7H), 6.99-6.94 (m, 2H), 5.06 (s, 2H), 3.18 (s, 1H), 2.97 (s, 1H), 2.60 (br s, 2H), 2.44 (br d, J=12.0 Hz, 1H), 2.17 (br d, J=12.0 Hz, 1H), 1.96 (br s, 0.5H), 1.80 (br d, J=0.5H), 1.73-1.43 (m, 8H); ¹³C NMR (100 MHz, CD₃OD) δ 157.7, 137.4, 137.3, 136.8, 128.5, 127.9, 127.4, 127.3, 126.8, 126.5, 124.5, 75.1, 74.9, 72.9, 72.6, 69.8, 60.4, 39, 38.9, 36.2, 35.7, 35.5, 34.3, 34.2, 33.5, 32.2, 27.3, 26.7, 21, 14.1 ppm. HRMS: M−H⁻. found 363.1970. C₂₄H₂₈O₃—H⁻ requires 363.1966.

### 4-(4-Benzyloxyphenyl)tricyclo[3.3.1.1^{3,7}]-decane-1-methanol (4a/b)

To a solution of 3a/b (1.27 g, 3.50 mmol) in dry THF (20 mL) at −78° C. was added NaCNBH₃ (1.100 g, 17.5 mmol). The reaction stirred for 30 min, and then BF₃-Et₂O (2.5 mL, 17.5 mmol) was added dropwise. The solution was warmed to room temperature and stirred overnight. The reaction was cautiously quenched with water (10 mL) and the resultant mixture extracted several times with ethyl acetate. The combined organic layers were washed successively with saturated aqueous sodium bicarbonate, water, and brine. The combined organic layers were dried (MgSO₄) and concentrated. The residue was purified by column chromatography (SiO₂, hexanes-ethyl acetate=4:1) to give 4a/b (630 mg, 52%) as a colorless oil which solidified upon standing. NMR spectroscopy revealed this to be a mixture of two stereoisomers (1:1.2 ratio by integration). mp 112-114° C.; ¹H NMR (400 MHz, CDCl₃) δ 7.65-7.23 (m, 7H, ArH), 6.98-6.92 (m, 2H, ArH), 5.06 (s, 2H, CH₂Ph), 3.30 (s, 0.9H, CH₂OH), 3.11 3.30 (s, 1.1H, CH₂OH), 2.92 and 2.89 (2×s, total 1H), 2.53 (br s, 2H), 1.93-1.80 (m, 4H), 1.75 and 1.70 (ABq, J=13.2H, 2H) 1.62-1.52 (m, 7H), 1.45 (d, J=12.4 Hz, 1H), 1.35-1.25 (m, 2H); ¹³C NMR (100 MHz, CDCl₃) δ 156.5, 153.5, 137.3, 136.1, 128.6, 127.9, 127.8, 127.7, 127.6, 127.5, 114.5, 114.4, 73.6, 73.5, 70.0, 45.8, 45.7, 40.4, 39.3, 39.2, 38.5, 34.5, 34.2, 33.4, 31.4, 31.1, 30.9, 28.0, 27.7 ppm. HRMS: M+NH₄⁺. found 366.2408. C₂₄H₂₈O₂+NH₄⁺ requires 366.2428.

### 4-(4-Hydroxyphenyltricyclo[3.3.1.1^{3,7}]-decane-1-methanol (5a/b)

To a solution of 4a/b (570 mg, 1.64 mmol) in methanol (15 mL) was added 10% Pd/C (349 mg, 3.28 mmol). The mixture was stirred under a balloon filled with H₂ at room temperature for 12 h. The reaction mixture was filtered through a pad of celite, dried (MgSO₄), and concentrated. The residue was purified by column chromatography (SiO₂, hexane-ethyl acetate 4:1) to give 5a/b (350 mg, 83%) as a colorless solid. NMR spectroscopy revealed this to be a mixture of two stereoisomers (˜1:1.8 ratio by integration). mp 148-150° C.; ¹H NMR (400 MHz, CD₃OD) δ 7.17-7.11 (m, 2H), 6.77-6.69 (m, 2H), 3.18 (s, 1.3H), 2.99 (s, 0.7H), 2.86-2.79 (m, 1H), 2.50-2.43 (m, 2H) 1.90-1.86 (m, 1H), 1.86-1.79 (m, 2H), 1.75-1.64 (m, 3H), 1.60-1.50 (m, 3H), 1.46-1.40 (m, 1H), 1.31-1.25 (m, 1H) ppm; ¹³C NMR (100 MHz, CD₃OD) δ 155.7, 135.8, 128.8, 128.6, 115.9, 73.8, 73.6, 47.1, 46.9, 41.6, 40.5, 40.4, 39.7, 35.5, 35.3, 34.5, 32.6, 32.5, 32.4, 29.6, 29.3 ppm. HRMS: M+NH₄⁺. found 276.1940. C₁₇H₂₂O₂+NH₄⁺ requires 276.1958.

### exo-7-[4-(t-Butyldimethylsilyloxy)phenyl]-1,1-dichloro-spiro[3.5]nonan-2-one (7) and endo-7-[4-(t-Butyldimethyl-silyloxy)phenyl]-1,1-dichloro-spiro[3.5]nonan-2-one (8)

To a solution of 6 (1.0 g, 3.3 mmol) in anhydrous ether (15 mL) under N₂ was added granulated Zn (0.648 g, 9.92 mmol). To the solution was added slowly via a syringe trichloroacetyl chloride (0.6 mL, 0.901 g, 9.92 mmol). The mixture was agitated in an ultrasonic bath for 1 h, and then heated with stirring at 45° C. for 3 h. After cooling to room temperature, the mixture was filtered through a pad of Celite and the filtrate was diluted with ether. The ether layer was then washed with saturated aqueous NH₄C, followed by saturated aqueous NaHCO₃ and finally by brine. The combined organic layers were dried (Na₂SO₄) and concentrated. The residue was purified by column chromatography (SiO₂, hexanes-ethyl acetate=10:1) to afford a mixture of stereoisomers 7 and 8 (0.712 g, 52%) as colorless oil. ¹H NMR (300 MHz, CDCl₃) δ 7.09 (m, 2H), 6.88-6.66 (m, 2H), 3.03 (s, 2H), 2.57 (q, J=6.8 Hz, 1H), 2.46-2.29 (m, 2H), 2.10-1.64 (m, 6H), 0.99 (m, 9H), 0.20 (s, 6H); ¹³C NMR (75 MHz, CDCl₃) δ 194.1, 154.1, 138.8, 127.8, 120.1, 91.6, 54.9, 45.3, 41.9, 35.4, 31.43, 25.9, 18.4, −4.20. The mixture was used in the following reaction without further characterization.

### 7-[4-(t-Butyldimethylsilyloxy)phenyl]spiro[3.5]nonan-2-one (9)

To a solution of 7/8 (0.300 mg, 0.726 mmol) in glacial acetic acid (10 mL) under N₂ was added in one portion granulated Zn (0.648 g, 9.92 mmol) and the mixture was heated to 70° C. for 16 h. After cooling to room temperature, the mixture was filtered through a pad of Celite to remove Zn residue. The filtrate was treated with water (30 mL) and extracted several times with ethyl acetate. The combined organic layers were washed with 1M aqueous NaOH, followed by brine, dried (Na₂SO₄) and concentrated. The residue was purified by column chromatography (SiO₂, hexanes-ethyl acetate=20:1) to afford 9 (0.200 g, 80%) as a colorless oil along with small amount of TBS deprotected phenol. ¹H NMR (400 MHz, CD₃OD) δ 7.07 (d, J=8.3 Hz, 2H), 6.74 (d, J=8.3 Hz, 2H), 2.80 (s, 2H), 2.75 (s, 2H), 2.51-2.40 (m, 1H), 1.86-1.70 (m, 6H), 1.47 (ddd, J=15.4, 12.3, 6.5 Hz, 2H), 0.98 (s, 9H), 0.17 (s, 6H); ¹³C NMR (100 MHz, CD₃OD) δ 212.9, 157.5, 131.2, 123.5, 118.6, 61.5, 58.6, 46.6, 41.1, 35.6, 28.8, −1.7. The material was used in the next step without further characterization.

### 7-(4-Hydroxyphenyl)spiro[3.5]nonan-2-one (10)

To a solution of 9 (0.200 g, 0.580 mmol) in methanol (10 mL) at rt was added dropwise via a syringe HF-pyridine complex (65% HF, 0.2 mL, 5.8 mmol). The reaction mixture was stirred at room temperature for 16 h. The mixture was quenched with water and the methanol was evaporated under vacuum. The resultant mixture was extracted several times with ethyl acetate. The combined organic layers were dried (Na₂SO₄) and concentrated to afford 10 (0.098 g, 73%) as a white solid; mp 150-154° C. mp 150-154° C.; ¹H NMR (400 MHz, CD₃OD) δ 7.03 and 6.70 (AA′BB′, J_{AB}=8.4 Hz, 4H), 2.82 (s, 2H), 2.77 (s, 1H), 2.45 (br m, J=12.2 Hz, 1H), 1.86-1.75 (m, 6H), 1.52-1.41 (m, 2H); ¹³C NMR (100 MHz, CD₃OD) δ 210.5, 156.4, 139.2, 128.6, 116.0, 58.9, 56.0, 44.0, 38.6, 33.1, 31.0 ppm. HRMS: M+H⁺. found 231.1369. C₁₅H₁₈O₂+H⁺ requires 231.1380.

### 7-(4-Hydroxyphenyl)spiro[3.5]nonan-2-ol (11)

To a solution of 10 (98 mg, 0.426 mmol) in methanol (10 mL) at 0° C. was added solid CeCl₃-7H₂O (167 mg, 0.449 mmol). After stirring for 10 min, solid NaBH₄ (18 mg, 0.468) was added. The mixture was warmed to room temperature and stirred for 3 h. Ice cold water (20 mL) was added to quench the reaction and then methanol was evaporated under reduced pressure. The residue was extracted several times with ethyl acetate and the combined organic layers were washed with brine, dried (Na₂SO₄) and concentrated. The reside was purified by column chromatography (SiO₂, hexanes-ethyl acetate=4:1) to give 11 (0.085 g, 86%) as a colorless solid. mp 194-195° C.; ¹H NMR (400 MHz, CD₃OD) δ 6.97 and 6.67 (AA′BB′, J_{AB}=8.3 Hz, 4H), 4.21 (pentet, J=7.3 Hz, 1H), 2.42-2.28 (m, 2H), 2.14-2.06 (m, 1H), 1.76-1.63 (m, 5H), 1.57-1.42 (m, 4H); ¹³C NMR (100 MHz, CD₃OD) δ 156.4, 139.7, 128.6, 116.1, 63.7, 45.5, 44.4, 42.2, 42.1, 38.6, 32.4, 32.2, 32.1 ppm. HRMS: M−H⁺. found 231.1391. C₁₅H₂₀O₂—H⁺ requires 231.1391.

### 7-[4-(t-Butyldimethylsilyloxy)phenyl)-1,5-dimethylbicyclo[3.3.0]oct-6-en-3-one (13)

To a solution of the 1-bromo-4-(t-butyldimethylsilyloxy)benzene (569 mg, 1.98 mmol) in dry THF (20 mL) at −78° C. under N₂ was added a solution of n-butyl lithium (1.1 mL, 2.0 M, 2.2 mmol). The mixture was stirred for 1 h. To the reaction mixture was added a solution of mono-ketal 12 (500 mg, 1.98 mmol) in dry THF (15 mL) at −78° C., and the mixture stirred for 3 h. The mixture was warmed to room temperature, quenched with water (25 mL), and the mixture was partitioned with CH₂Cl₂. The combined organic layers were dried (MgSO₄) and concentrated. The crude product was dissolved in a mixture of benzene (15 mL) and acetone (10 mL), and p-toluenesulfonic acid (101 mg, 0.586 mmol) was added. The mixture was heated to 70° C. for 16 h, cooled to room temperature and diluted with ethyl acetate. The mixture was washed with saturated aqueous NaHCO₃, followed by brine and the combined aqueous layers were extracted with ethyl acetate. The combined ethyl acetate layers were dried (MgSO₄) and concentrated. Purification of the residue by flash chromatography (SiO₂, hexanes-ethyl acetate=4:1) gave (±)-13 as a colorless oil (160 mg, 23%). ¹H NMR (300 MHz, CDCl₃) δ 7.08 and 6.60 (AA′BB′, J_{AB}=8.5 Hz, 4H), 5.67 (s, 1H, C═CH), 2.54 (dd, J=1.8, 15.6 Hz, 1H), 2.49 (d, J=15.6 Hz, 1H), 2.36-2.21 (m, 3H), 2.10-1.90 (m, 3H), 1.12 (s, 3H), 0.95 (s, 3H), 0.79 (s, 9H), 0.00 (s, 6H); ¹³C NMR (75 MHz, CDCl₃) δ 218.4, 155.3, 139.5, 132.4, 129.3, 126.7, 120.0, 54.3, 53.7, 51.4, 49.7, 47.4, 46.1, 25.7, 21.9, 18.2, −4.4 ppm.

### 7-(4-Hydroxyphenyl)-1,5-dimethylbicyclo[3.3.0]oct-6-en-3-one (14)

To a solution of (±)-13 (155 mg, mmol) in dry THF (10 mL) and pyridine (1 mL) in a Teflon reaction vessel was added 65% HF-pyridine (0.1 mL,). The reaction mixture was stirred a room temperature for 20 h, at which time TLC (hexanes-ethyl acetate=3:1) indicated disappearance of the starting material. The mixture was diluted with ethyl acetate, quenched with 10% aqueous HCl (5 mL), and the layers separated. The organic layer was washed with brine, and the combined aqueous layers were further extracted with ethyl acetate. The combined ethyl acetate layers were dried (MgSO₄) and concentrated to afford (±)-14 (87 mg, 83%) as a colorless solid. ¹H NMR (400 MHz, CD₃OD) δ 7.28 and 6.72 (AA′BB′, J_{AB}=8.6 Hz, 4H), 5.86 (s, 1H, C═CH), 2.76-2.66 (m, 2H), 2.51-2.23 (m, 4H), 1.23 (s, 3H), 1.16 (s, 3H) ppm.

### (±)-endo-7-(4-Hydroxyphenyl)-1,5-dimethylbicyclo[3.3.0]oct-6-en-3-ol and (±)-exo-7-(4-Hydroxyphenyl)-1,5-dimethylbicyclo[3.3.0]oct-6-en-3-ol (15)

To a solution of (±)-14 (74 mg, 0.31 mmol) in anhydrous THF (10 mL) at 0° C. under N was added in small portions solid LiAlH₄ (55 mg, 1.4 mmol). The mixture was stirred at 0° C. for 2 h, and then 1-120 (1 mL) was added dropwise very slowly until effervescence ceased. Dilute aqueous NaOH (1 mL) was added, followed by H₂O (50 mL). The mixture was extracted several times with ethyl acetate, the combined extracts were dried (MgSO₄) and the solvent evaporated to afford a colorless solid (65 mg, 86%). The ¹H NMR spectrum of this indicated this to be a mixture of endo- and exo-alcohol stereoisomers. ¹H NMR (400 MHz, CD₃OD) δ 7.25-7.18 (m, 2H, ArH), 6.70 (d, J=8.4 Hz, 2H, ArH), 5.83 (s, 0.65H, H-6), 5.63 (s, 0.35H, H-6), 4.26 (p, J=6.8 Hz, H-3endo), 4.06 (tt, J=6.8, 8.8 Hz, H-3exo), 2.69 (d, J=15.8 Hz, 0.65H), 2.08-1.98 (m, 1H), 1.95-1.87 (m, 1.5H), 1.85-1.74 (m, 1.8H), 1.58-1.44 (m, 1.2H), 1.40-1.30 (m, 1.?H), 1.17 (s, 1.2H, CH₃), 1.09 (s, 3.3H, CH₃), 1.017 and 1.007 (2×s, 2.8H, CH₃); ¹³C NMR (100 MHz, CD₃OD) δ 157.6, 139.3, 137.6, 135.1, 133.1, 129.8, 127.8, 116.0, 72.2 (71.6), 57.2 (56.9), 53.3, 52.3, 52.2, 51.4, 49.8, (25.8) 24.0, 23.7 (23.3) ppm (diastereomeric signals in brackets).

### 4.2 Biological Evaluation

### 4.2.1 TR-FRET Ligand Binding Displacement Assay

Competitive ligand binding analyses were done using the Thermo Fisher Scientific LanthaScreen™ assay. A ‘donor’ terbium-labeled antibody for the GST-binding domain was bound to a GST-ERβ construct, containing the ligand binding domain (LBD). A fluorescently-labeled tracer molecule containing 17-β-Estradiol (Fluoromone E2) was bound to the GST-ERβ-LBD. Excitation of the ‘donor’ antibody resulted in the transfer of energy to the ‘acceptor’ tracer molecule. Displacement of the tracer molecule was measured after introduction of the compounds and then calculated using a ratio of the fluorescein-labeled ‘acceptor’ (520 nm) and terbium ‘donor’ (495 nm) emission values. A 10-point titration was performed using 1:2 serial dilutions of compounds starting at 1000 nM and the analyses were performed at a final and fixed DMSO concentration of 1%. Emission ratios were normalized to an assay control of 17-β-Estradiol with an IC₅₀ of 0.497 nM. Data were analyzed using GraphPad Prism® 6 for Windows, ver. 6.07 (Jun. 12, 2015). An IC₅₀ for each compound was calculated using a nonlinear least-squares fit (normalized variable slope analysis) of equation 1. Standard deviation values are from this fitting process.

$$y = 100 / ( 1 + {10}^{( logIC 50 - x ) * Hillslope )} ) \qquad ( 1 )$$

Note that because GraphPad Prism® 6 provides standard deviations (SDs) of Log(IC₅₀), the following correction is needed to obtain standard deviations:

$$SD of {IC}_{50} = ( ( ( SD of Log {IC}_{50} ) / 0.434 ) * {IC}_{50} ) \qquad ( 2 )$$

### 4.2.2 Cell-Based Agonist and Antagonist Assays

Agonistic and antagonistic activity was measured using ERα and ERβ cell-based assay kits supplied by Indigo Biosciences. The kit utilizes non-human cells engineered to express the full-length human Estrogen Receptor (ER) 1 (NR3A1). Cells contain a luciferase reporter gene that is linked to an ERα or ERβ responsive promoter that can quantify activity changes of the receptor. The change of ER activity is dependent on the agonist or antagonist properties of the added compound. A luciferase detection agent is used to quantify the luminescence intensity of the luciferase expression induced by the ER, which is measured using a SpectraMax M5 plate reader. Ligand stock solutions were prepared in DMSO and diluted to their final concentration using the Compound Screening Medium (CSM) that was provided with the kit. The DMSO concentration of each stock solution was kept below the assay limit of 0.4%. Assays were performed according to the kit instructions with the addition of a vehicle control for both the agonist and antagonist assay. Briefly, cells were removed directly from the freezer, where they were diluted in the Cell Recovery Media (CRM) provided with the kit. They were immediately placed in a warming bath for 5 minutes at 37° C. The cell suspension was divided in half and estradiol (E₂) was added to one-half of the cells for the antagonist assay, while the other half of the cells that contained no E₂ were used for the agonistic assay. Assays were performed in duplicates and two trials with ERβ were done for 5a/b and (±)-11. Cells were plated and the compound of interest was added. Plates were then incubated for 24 hours at 37° C. with 5% CO₂. Cell media was removed and Luciferin Detection Reagent was added to measure the luminescence. The data were normalized to E₂ using GraphPad Prism and fitted to equation 3:

$$y = {Y}_{L} + ( {Y}_{H} - {Y}_{L} ) ( 1 + {10}^{( ( logEC 50 - x ) * HillSlope )} ) \qquad ( 3 )$$

where Y_{L} and Y_{H} were low and high plateau values, respectively. For fitting, Y_{L} was constrained to 0. Standard deviations were calculated according to eqn. 2.

### 4.2.3 Cytochrome P450 Binding Measurements

A screen of Cytochrome p450 inhibition was performed using the Promega P450-Glo™ Screening Systems. The Screening Systems' manual instructions were followed for CYP2C9, CYP2D6, or CYP3A4 enzymes with Luciferin-H, Luciferin-ME EGE or Luciferin-PPXE as substrates, respectively. The components of the kits, i.e., membrane enzymes, control enzymes, substrates and potassium phosphate buffer and or TRIS buffer were combined according to the System-manual's recommended final concentrations. Reactions were pre-incubated in white 96-well flat-bottom plates at 37° C. for 10 min before starting reactions with 2×NADPH regeneration buffer. The 96-well plates were incubated according to the recommended times in the linear range for each enzyme/substrate reaction. The reactions were stopped using 2× Luciferin Detection Reagent. After 20 minutes, the luciferase product was detected using a SpectraMax M5 Molecular Devices plate reader on the end-point luminescence setting. Positive control inhibitors for CYP2C9, CYP2D6, and CYP3A4 enzymes were assayed at a final concentration of 10 μM sulfaphenazole, 1 μM quinidine and 5 μM ketoconazole, respectively. Serial dilutions of 5a/b and (±)-11 were assayed at final concentrations of 62.5, 31.25, 15.125, 7.812, 3.906, 1.953, 0.977, 0.488 μM. Inhibition data were analyzed using GraphPad Prism® 6 for Windows, ver. 6.07 (Jun. 12, 2015) software. Raw data were normalized against the control enzyme and untreated CYP enzyme means and then analyzed through non-linear regression using log (inhibitor) vs normalized dose-response curves to assess IC₅₀s. Standard deviations were calculated from Best-fit values. Data were normalized to vehicle and positive controls (sulfaphenazole for CYP2C9, quinidine for CYP2D6, and ketoconazole for CYP3A4) and nonlinear square fits of the data were conducted using Prism 6 (GraphPad).

### 4.3.5 ERβ Docking Studies

Docking of 5b and 11 into the binding pocket of human ERβ (PDB ID: 2JJ3) was done using the Glide function in Schrodinger Maestro 12.5.¹⁶ Preprocess, review and modify, and refine steps were performed. The conditions set for the preprocess were: assign bond orders through the use of the CCD database, add hydrogens, create zero-order bonds to metals, create disulfide bonds, and generate “het” states using Epik with a pH between 7.0+/−2.0. Due to the fact that the lobes of ER-β are the same, in review and modify, Chain B was deleted. To refine the preprocessed protein, the hydrogen bond assignment, the sample water orientation, and the hydrogens were minimized. The minimization step converged heavy atoms to an RMSD of 0.30 Å. The force field used for the minimization was OPLS3e. LigPrep¹⁷ was used to properly prepare the structures of 5b and 11 for docking. These molecules were prepared with the OPLSe force field and generated possible states at a pH of 7.0+/−2.0 using the ionizer function. Furthermore, the desalt feature as well as the generate tautomer option were selected. The rest of the settings were set to defaults. Standard precision docking was used, with flexible ligand sampling. The resulting figures (FIG. 3) indicate interactions with key residues and the orientation of the molecules in the active site.

### 4.3.6 Cytochrome P450 Metabolism Predictions

Using the P450 Perform Calculations function of Schrodinger software, the previously prepared ligands (5a/b; 11) were analyzed to predict susceptibility to CYP450 metabolism. The CYP450 isoform that was selected was 3A4, 2C9 or 2D6, which calculates predicted intrinsic reactivity. The more positive the number, the more reactive that atom is. The 2C9 and 2D6 calculations also include induced fit docking and subsequent measurement of Fe-accessibility. This is the natural logarithm of the number of poses for an atom within 5 angstroms of the reactive heme Fe atom.

### REFERENCES AND NOTES

1. J. F. Couse, J. Lindzey, K. Grandien, J. Gustafsson, K. S. Korach, Tissue Distribution and Quantitative Analysis of Estrogen Receptor-□ (ER□) and Estrogen Receptor-□ (ERL□) Messenger Ribonucleic Acid in the Wild-Type and ERa-Knockout Mouse, Endocrinology 1997, 138, 4613-4621.
2. B. Potter, S. Schrager, J. Dalby, E. Torell, A. Hampton, A. Menopause, Prim. Care 2018, 45, 625-641.
3. (a) J. E. Rossouw, G. L. Anderson, R. L. Prentice, A. Z. LaCroix, C. Kooperberg, M. L. Stefanick, R. D. Jackson, S. A. Beresford, B. V. Howard, K. C. Johnson, J. M. Kotchen, J. Ockeen, Risks and benefits of estrogen plus progestin in healthy postmenopausal women, JAMA 2002, 288, 321-333. (b) K. F. Koehler, L. A. Helguero, L. A. Haldosen, M. Warner, J. A. Gustafsson, J. A. Reflections on the discovery and significance of estrogen receptor beta, Endocr. Rev. 2005, 26, 465-477.
4. S. Ali, R. C. Coombes, Estrogen receptor alpha in human breast cancer: Occurrence and significance, J. Mammary Gland Biol. Neoplasia 2000, 5, 271-281.
5. (a) D. M. Tanenbaum, Y. Wang, S. P. Williams, P. B. Sigler, Crystallographic comparison of the estrogen and progesterone receptor's ligand binding domains. Proc. Natl. Acad. Sci. U.S. A. 1998, 95, 5998-6003. (b) A. M. Brzozowski, A. C. Pike, Z. Dauter, R. E. Hubbard, T. Bonn, O. Engstrom, L. Ohman, G. L. Greene, J. A. Gustafsson, M. Carlquist, Molecular basis of agonism and antagonism in the oestrogen receptor. Nature 1997, 389, 458-461.
6. B. H. Norman, J. A. Dodge, T. I. Richardson, P. S. Borromeo, C. W. Lugar, S. A. Jones, K. Chen, Y. Wang, R. J. Durst, C. Barr, C. Montrose-Rafizdeh, H. E. Osborne, R. M. Amos, S. Guo, A. Boodhoo, V. Krishnan, Benzopyrans are selective Estrogen receptor b agonists with novel activity in models of benign prostatic hyperplasia, J. Med. Chem. 2006, 49, 6155-6157.
7. V. M. Carroll, M. Jeyajumar, K. E. Carlson, J. A. Katzenellenbogen, Diarylpropionitrile (DPN) enantiomers: Synthesis and evaluation of estrogen receptor b-selective ligands, J. Med. Chem. 2011, 55, 528-537.
8. K. L. I. S. Perera, A. M. Hanson, S. Lindeman, A. Imhoff, X. Lu, D. S. Sem, W. A. Donaldson, Synthesis and evaluation of 4-cyclhepthyphenols as selective estrogen receptor-b agonists (SEBAs), Eur. J. Med Chem. 2018, 157, 791-804.
9. A. M. Hanson, K. L. I. S. Perera, J. Kim, R. K. Pandey, N. Sweeney, X. Lu, A. Imhoff, A. C. Mackinnon, A. J. Wargolet, R. M. Van Hart, K. M. Frick, W. A. Donaldson, D. S. Sem, D. S. A-C Estrogens as potent and selective estrogen receptor-beta agonists (SERBAs) to enhance memory consolidation under low-estrogen conditions, J. Med Chem. 2018, 61, 4720-4738.
10. A. M. Fleischer, J. C. Schalk, E. A. Wetzel, A. M. Hanson, D. S. Sem, W. A. Donaldson, K. M. Frick, Long-term oral administration of a novel estrogen receptor beta agonist enhances memory and alleviates drug-induced vasodilation in young ovariectomized mice, Horm. Behav. 2021, 130, 104948.
11. (a) Y. Endo, T. Iijima, Y. Yamakoshi, M. Yamaguchi, H. Fukasawa, K. Shudo, Potent Estrogenic agoists pering dicarba-closo-dodecaborane as a hydrophobic pharmacophore, J. Med Chem. 1999, 42, 1501-1504. (b) Y. Endo, T. Iijima, Y. Yamakoshi, H. Fukasawa, C. Miyaura, M. Inada, A. Kubo, A. Itai, Potent estrogen agonists based on carborane as a hydrophobic skeletal structure. A new medicinal application of boron clusters, Chem. Biol. 2001, 8, 341-355. (c) K. Ohta, T. Ogawa, A. Oda, A. Kaise, Y. Endo, Design and synthesis of carborane-containing estrogen receptor-beta (ERb)-selective ligands, Boorg. Med Chem. Lett. 2015, 25, 4174-4178. (d) D. Sedlak, T. A. Wilson, W. Tjarks, H. S. Radomska, H. Wang, J. N. Kolla, Z. J. Lesnikowski, Al Spicakova, T. Ali, K. Ishita, L. H. Rakotondriabe, S. Vibhute, D. Wang, P. Anzenbacher, C. Bennett, P. Bartunek, C. C. Coss, Structure-Activity Relationship of para-Carborane Selective Estrogen Receptor b Agonists, J. Med Chem. 2021, 64, 9330-9353.
12. L. Wanka, K. Iqbal, P. R. Schreiner, The lipophilic bullet hits the targets: Medicinal chemistry of adamantane derivatives, Chem. Rev. 2013, 113, 3516-3604.
13. P. S. Manchand, R. L. Cerruti, J. A. Martin, C. H. Hill, J. H. Merrett, E. Keech, R. B. Belshe, E. V. Connell, I. S. Sim, Synthesis and antiviral activity of metabolites of rimantadine, J. Med Chem. 1990, 33, 1992-1995.
14. CCDC 2088848 (11) contains the supplementary crystallographic data for this paper. These data are provided free of charge by The Cambridge Crystallographic Data Centre.
15. (a) J. A. Hyatt, P. W. Raynolds, Ketene Cycloadditions, Org. React. 1994, 45, 159-646. (b) J. P. Depres, P. Delair, J. F. Poisson, A. Kanazawa, A. E. Green, Diverse natural products from dichlorocyclobutanones: An evolutionary tale, Acc. Chem. Res. 2016, 49, 252-261.
16. (a) F. Wu, K. Matsuyama, WO Patent 2013000275, 2013; Chem. Abstr. 1985, 65, 2870. N. Feuerbacher, F. Voegtle, J. Windscheidt, E. Poetsch, M. Nieger, Synthesis of rodlike dispirohydrocarbon skeletons for new liquid crystal compounds, Synthesis 1999, 117-120. (b) B. M. Trost, M. J. Bogdanowicz, New synthetic reactions. X. Versatile cyclobutanone (spiroannelation) and g-butyrolactone (lactone annelation) synthesis, J. Am. Chem. Soc. 1973, 95, 5321-5334.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

### Example 2

trans-4-(4-(Fluoromethyl)cyclohexyl)phenol. To a stirred solution of compound trans-4-(4-(hydroxymethyl)cyclohexyl)phenol¹ (0.065 g, 0.315 mmol) in CH₂Cl₂ (12 mL) was added bis(2-methoxyethyl)aminosulfur trifluoride (0.09 mL, 0.473 mmol) in CH₂Cl₂ (3 mL) at −78° C. The mixture was stirred under N₂ and gradually warmed to room temperature. On completion, saturated NaHCO₃ (10 mL) was poured into the mixture. After C02 evolution ceased the mixture was extracted several times with CH₂Cl₂, the combined extracts were dried (Na₂SO₄) and concentrated. The residue was purified by column chromatography (SiO₂, hexanes-ethyl acetate=4:1) to give trans-4-(4-(fluoromethyl)cyclohexyl)phenol (0.037 g, 56%) as a colorless solid. mp 103-109° C.; ¹H NMR (400 MHz, CD₃OD) δ 7.00 and 6.68 (AA′BB′, J_{AB}=8.5 Hz, 4H), 4.23 (dd, J_{H-F}=48.1 Hz, J_{H-H}=7.4 Hz, 2H), 2.37 (tt, J=12.3, 2.8 Hz, 1H), 1.90-1.80 (m, 4H), 1.78-1.59 (m, 2H), 1.52-1.37 (m, 2H), 1.24-1.09 (m, 2H); ¹³C NMR (100 MHz, CD₃OD) S 156.5, 139.7, 128.7, 116.1, 90.2, 89.5 (d, J_{C-F}=166.5 Hz), 45.0, 39.8 (d, J_{C-F}=18.0 Hz), 35.1, 30.1, 29.9; ¹⁹F NMR (376 MHz, CD₃OD) δ −224.5 (dt, J_{H-F}=17.3, 47.8 Hz) ppm.

1-(4-(Benzyloxy)phenyl)-4-(trifluoromethyl)cyclohexan-1-ol. To a solution of 4-trifluoromethylcyclohexanone (0.200 mg, 1.20 mmol) in anhydrous THF (15 mL) at 0° C. under N₂ was slowly added a solution of (4-(benzyloxy)phenyl)magnesium bromide (0.8 M in THF, 2.3 mL, 1.81 mmol). The mixture was stirred at 0° C. for 30 min and then at room temperature for 16 h. The solution was cooled to 0° C. and quenched with water (30 mL), followed by 1 M aqueous HCl (30 mL). The mixture was extracted several times with ethyl acetate and the combined organic layers were washed with brine, dried (Na₂SO₄), and concentrated. The crude residue was purified by column chromatography (SiO₂, hexanes-ethyl acetate=4:1) to give 1-(4-(benzyloxy)phenyl)-4-(trifluoromethyl)cyclohexan-1-ol (0.260 g, 62%) as a mixture of cis- and trans-diastereomers as a yellow waxy-solid. This material was carried forward in the next step without further purification. ¹H NMR (400 MHz, CDCl₃) δ 7.52-7.36 (m, 6H), 7.02 (d, J=12.0 Hz, 2H), 5.09 (s, 2H), 2.48-2.40 (m, 3H), 2.32-1.80 (m, 2H), 1.72 (dt, J=3.4, 12.3 Hz, 2H), 1.55-1.47 (m, 2H); ¹³C NMR (100 MHz, CDCl₃) δ 158.0, 136.9, 128.6, 128.0, 127.6, 127.5, 127.0, 114.9, 72.2, 69.9, 39.8 (q, J_{C-F}=26.0 Hz), 35.8, 21.9 ppm.

trans-1-(Benzyloxy)-4-(4-trifluoromethylcyclohexyl)benzene. To a solution 1-(4-(benzyloxy)phenyl)-4-(trifluoromethyl)cyclohexan-1-ol (0.260 g, 0.742 mmol) and triethylsilane (0.20 mL, 1.5 mmol) in dry CH₂Cl₂ (30 mL) at 0° C. under N₂ was added slowly via syringe BF₃-Et₂ O (0.20 mL, 1.5 mmol). After the addition was completed the reaction mixture was brought to room temperature and stirred for 3 h. A saturated aqueous NaHCO₃ solution (20 mL) was added, the layers were separated, and the aqueous layer was extracted several times with CH₂Cl₂. The combined organic layers were washed with brine, dried (Na₂SO₄) and concentrated. The residue was purified by column chromatography (SiO₂, hexanes-ethyl acetate=20:1) to give trans-1-(benzyloxy)-4-(4-trifluoromethylcyclohexyl)benzene (0.150 g, 60%) as an off-white solid; mp=100-105° C. ¹H NMR (400 MHz, CDCl₃) δ 7.49-7.34 (m, 5H), 7.17 (d, J=8.6 Hz, 2H), 6.98 (d, J=8.6 Hz, 2H), 5.08 (s, 2H), 2.59-2.47 (m, 1H), 2.20-1.90 (m, 5H), 1.57-1.43 (m, 4H); ¹³C NMR (100 MHz, CDCl₃) δ 157.2, 138.8, 137.1, 128.4, 128.0, 127.6, 127.5, 114.7, 70.0, 42.6, 41.5 (q, J_{C-F}=26.3 Hz), 32.7, 29.1, 25.3; ¹⁹F NMR (376 MHz, CDCl₃) δ −73.7 (d, J=8.3 Hz).

trans-4-(4-(Trifluoromethyl)cyclohexyl)phenol. To a solution of trans-1-(benzyloxy)-4-(4-trifluoromethylcyclohexyl)benzene (0.140 g, 0.419 mmol) in methanol (10 mL) was added 10% Pd/C (45 mg, 0.042 mmol, 10 mol %). The mixture was stirred under a balloon filled with H₂, at room temperature, for 12 h. The reaction mixture was filtered through a pad of celite and concentrated to afford trans-4-(4-(trifluoromethyl)cyclohexyl)phenol (0.090 g, 88%) as a brown solid; mp=95-100° C.; ¹H NMR (400 MHz, CD₃OD) δ 6.99 (d, J=8.2 Hz, 2H), 6.69 (d, J=8.2 Hz, 2H), 2.42-2.33 (m, 1H), 2.17-2.06 (m, 1H), 2.04-1.80 (m, 4H), 1.74-1.64 (m, 1H), 1.50-1.35 (m, 3H); ¹³C NMR (101 MHz, CD₃OD) δ 156.5, 138.7, 128.6, 116.0, 43.9, 42.5 (q, J_{C-F}=26.0 Hz), 33.9, 30.3, 26.4; ¹⁹F NMR (376 MHz, CD₃OD) δ −75.2 (d, J=8.3 Hz).

2-Fluoro-4-(4-hydroxycyclohexyl)phenol. To a solution of 4-(3-fluoro-4-hydroxyphenyl)cyclohexan-1-one² (0.033 g, 0.159 mmol) in anhydrous methanol (10 mL) was added NaBH₄ (0.090 g, 2.38 mmol). The reaction was stirring at room temperature for 2 h and then diluted with water. The resulting mixture was extracted with ethyl acetate (2×15 mL) and combined extracts were concentrated. Purification of the residue by column chromatography (SiO₂, hexanes-ethyl acetate=13:7) gave 2-fluoro-4-(4-hydroxycyclohexyl)phenol (0.020 g, 61%) as a colorless solid. mp 179-186° C. ¹H NMR (400 MHz, CD₃OD) δ 6.91-6.85 (m, 1H), 6.83-6.74 (m, 2H), 3.60-3.52 (m, 1H), 2.39 (tt, J=12.0, 3.1 Hz, 1H), 2.05-1.96 (m, 2H), 1.88-1.79 (m, 2H), 1.52-1.20 (m, 4H); ¹³C NMR (100 MHz, CD₃OD) δ 152.8 (d, J_{C-F}=240 Hz), 144.0 (d, J_{C-F}=10 Hz), 140.3, 123.6, 118.5, 115.1 (d, J_{C-F}=10 Hz), 71.1, 44.0, 36.6, 33.8 ppm. HRMS: M−H⁺. found 209.0983. C₁₂H₁₅O₂F—H⁺ requires 209.0983.

4-(4-(Benzyloxy)-3-fluorophenyl)cyclohexan-1-one. To a solution of 4-(3-fluoro-4-hydroxyphenyl)cyclohexan-1-one (0.205 g, 0.984 mmol) in N,N-dimethylformamide (10 mL), was added benzyl bromide (0.219 g, 0.15 mL, 1.28 mmol) followed by potassium carbonate (0.177 g, 1.28 mmol). The mixture was heated at reflux for 6 h. After cooling to room temperature, the mixture was poured into ice-cold water and extracted with ethyl acetate (2×15 mL). The combined organic extracts were and washed with brine (3×15 mL), dried (Na₂SO₄) and concentrated. The residue was purified by column chromatography (SiO₂, hexanes-ethyl acetate=9:1) to give 4-(4-(benzyloxy)-3-fluorophenyl)cyclohexan-1-one (0.232 g, 79%) as a colorless solid. mp 55-60° C. ¹H NMR (400 MHz, CDCl₃) 7.49-7.29 (m, 5H), 7.02-6.88 (m, 3H), 5.11 (s, 2H), 2.95 (tt, J=12.2, 6.9 Hz, 1H), 2.51-2.43 (m, 3H), 2.22-2.13 (m, 2H), 1.92-1.79 (m, 2H); ¹³C NMR (100 MHz, CDCl₃) δ 211.0, 152.9 (d, J_{C-F}=245 Hz), 145.2 (d, J_{C-F}=11 Hz), 138.6, 136.7, 128.7, 128.2, 127.5, 122.2, 115.9, 114.7 (d, J_{C-F}=18 Hz), 71.6, 41.8, 41.3, 34.0 ppm. HRMS: M+Na⁺. found 321.1262. C₁₉H₁₉O₂F+Na⁺ requires 321.1269.

1-(Benzyloxy)-2-fluoro-4-(4-methylenecyclohexyl)benzene. A solution of n-butyllithium in hexane (2.5 M, 0.47 mL, 1.17 mmol) was slowly added to a stirring solution of methyltriphenyl-phosphonium bromide (0.556 g, 1.56 mmol) in dry THF (20 mL) at −10° C. After 20 min, a solution of 4-(4-(benzyloxy)-3-fluorophenyl)cyclohexan-1-one (0.232 g, 0.778 mmol) in dry THF (10 mL) was added dropwise. The reaction mixture was slowly warmed to room temperature and stirred overnight. The mixture was diluted with water (10 mL), extracted with ethyl acetate (2×25 mL), dried (Na₂SO₄) and concentrated. The residue was purified by column chromatography (SiO₂, hexanes-ethyl acetate=9:1) to give 1-(benzyloxy)-2-fluoro-4-(4-methylene-cyclohexyl)benzene (0.165 g, 72%) as a colorless solid. ¹H NMR (400 MHz, CDCl₃) 7.56-7.29 (m, 5H), 7.02-6.83 (m, 3H), 5.13 (s, 2H), 4.71 (s, 2H), 2.63 (tt, J=12.2, 3.3 Hz, 1H), 2.49-2.37 (m, 2H), 2.27-2.12 (m, 2H), 2.04-1.92 (m, 2H), 1.57-1.43 (m, 2H) ppm. ¹³C NMR (100 MHz, CDCl₃) δ 153.0 (d, J_{C-F}=250 Hz), 148.6, 144.9, 141.0, 137.0, 133.9 (d, J_{C-F}=20 Hz), 128.7, 128.2, 127.6, 122.3, 115.8, 114.9 (d, J_{C-F}=20 Hz), 107.8, 71.7, 43.3, 35.7, 35.2 ppm. HRMS: M+Na⁺. found 319.1472. C₂₀H₂₁OF+Na⁺ requires 319.1469.

(4-(4-(Benzyloxy)-3-fluorophenyl)cyclohexyl)methanol. A solution of 9-BBN in THF (0.5 M, 1.46 mL, 0.729 mmol) at 0° C., was added to a solution of 1-(benzyloxy)-2-fluoro-4-(4-methylene-cyclohexyl)benzene (0.108 g, 0.364 mmol) in THF (15 mL). The reaction mixture was slowly warmed to room temperature and stirred for 20 h. The mixture was again cooled to 0° C., followed by the sequential addition of hydrogen peroxide solution (30% in water, 0.20 mL) and 1N NaOH solution (0.50 mL). The resulting mixture was warmed to room temperature, stirred for 15 min and extracted with ethyl acetate (2×20 mL). The combined organic extracts were dried (Na₂SO₄) and concentrated. The residue was purified by column chromatography (SiO₂, hexanes-ethyl acetate=6:4) to give (4-(4-(benzyloxy)-3-fluorophenyl)cyclohexyl)methanol (0.025 g, 22%) as a colorless solid. This was determined to be a 1:2 mixture of cis- and trans-stereoisomers by ¹H NMR integration of the hydroxymethylene doublets (S 3.67 and 3.50 ppm respectively). ¹H NMR (400 MHz, CDCl₃) 7.50-7.28 (m, 5H), 7.01-6.80 (m, 3H), 5.11 (s, 2H), 3.67 (d, J=7.9 Hz, 0.7H), 3.50 (d, J=6.2 Hz, 1.3H), 2.60-2.50 (m, 0.3H), 2.47-2.36 (m, 0.7H), 1.98-1.33 (m, 8H), 1.17-1.03 (m, 1H); ¹³C NMR (100 MHz, CDCl₃) δ 153.0 (d, J_{C-F}=247 Hz), 144.8, 141.6, 137.0, 128.8, 128.2, 127.6, 122.4, 122.3, 115.9, 115.1, 114.9, 114.8 (d, J_{C-F}=16 Hz), 71.8, 68.7, 64.6, 43.7, 42.2, 40.2, 36.2, 33.9, 29.8, 29.3, 26.8 ppm.

2-Fluoro-4-(4-(hydroxymethyl)cyclohexyl)phenol. To a solution of (4-(4-(benzyloxy)-3-fluorophenyl)cyclohexyl)methanol (0.050 g, 0.159 mmol) in ethyl acetate (10 mL) was added 10% Pd/C (0.017 g, 10 mol %) and mixture was stirred for 12 h, at room temperature, under a balloon filled with H₂. The reaction mixture was filtered through a pad of celite and concentrated. The residue was purified by column chromatography (SiO₂, hexanes-ethyl acetate=3:2) to give 2-fluoro-4-(4-(hydroxymethyl)cyclohexyl)phenol 82 (0.018 g, 51%) as a colorless solid. This was determined to be a 1:2 mixture of cis- and trans-stereoisomers by ¹H NMR integration of the hydroxymethylene doublets (S 3.60 and 3.39 ppm respectively). ¹H NMR (400 MHz, CD₃OD) 6.92-6.85 (m, 1H), 6.84-6.76 (m, 2H), 3.60 (d, J=7.4 Hz, 0.7H), 3.39 (d, J=6.5 Hz, 1.3H), 2.55-2.33 (m, 1H), 1.94-1.34 (m, 8H), 1.15-1.01 (m, 1H); ¹³C NMR (100 MHz, CD₃OD) δ 154.0, 151.6, 143.9, 141.1, 123.5, 118.5, 115.0, 68.8, 64.4, 45.0, 41.3, 37.1, 35.2, 31.0, 30.4, 29.5, 27.8 ppm. HRMS: M−H⁺. found 223.1140. C₁₃H₁₇O₂F−H⁺ requires 223.1140.

### REFERENCES TO EXAMPLE 2

1) A. M. Hanson, K. L. I. S. Perera, J. Kim, R. K. Pandey, N. Sweeney, X. Lu, A. Imhoff, A. C. Mackinnon, A. J. Wargolet, R. M. Van Hart, K. M. Frick, W. A. Donaldson, D. S. Sem, “A-C Estrogens as Potent and Selective Estrogen Receptor-Beta Agonists (SERBAs) to Enhance Memory Consolidation under Low-Estrogen Conditions”, J. Med. Chem. 2018, 61, 4720-4738.
2) C. Benecke, T. Kukac, A. Ohlemacher, “New polymerizable liquid crystalline compounds”, WO 9852905, Nov. 26, 1998.

We claim:

1. A compound having a Formula I: 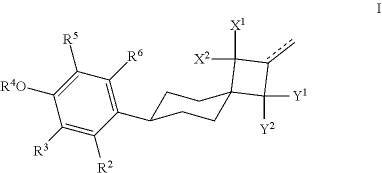
wherein X¹, X², Y¹, and Y² are independently selected from the group consisting of hydrogen, halogen, and hydroxyl;
optionally with the proviso that when X¹ and X² are halogen then Y¹ and Y² are hydrogen and optionally with the proviso that when Y¹ and Y² are halogen then X¹ and X² are hydrogen;
W is selected from the group consisting of hydrogen, hydroxyl, and oxo;
R², R³, R⁵, and R⁶ are independently selected from hydrogen, deuterium, and halogen; and
R⁴ is hydrogen or a hydroxyl protecting group.

2.-3. (canceled)

4. The compound of claim 1, wherein W is oxo, R⁴ is a hydroxyl protecting group, and R², R³, R⁵, R⁶ are hydrogen, the compound having a Formula I(a):

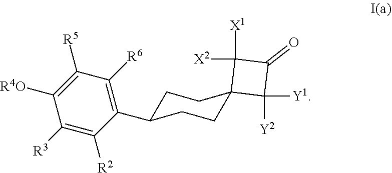

5.-7. (canceled)

8. The compound of claim 1, wherein X¹, X², Y¹, Y², R², R⁴, and R⁶ are hydrogen, the compound having a Formula I(b):

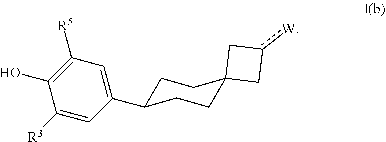

9.-11. (canceled)

12. The compound of claim 1, wherein W and R⁴ are hydrogen, the compound having a Formula I(c):

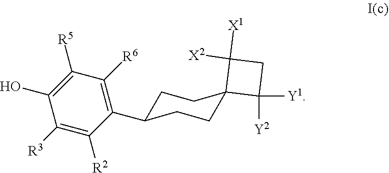

13.-14. (canceled)

15. A pharmaceutical composition comprising an effective amount of the compound of claim 1, or a pharmaceutically acceptable salt thereof, together with a pharmaceutical excipient, carrier, or diluent.

16. A method for treating a patient a disease or disorder associated with estrogen receptor β (ERβ) activity, the method comprising administering the pharmaceutical composition of claim 15.

17.-19. (canceled)

20. A compound having a Formula II: 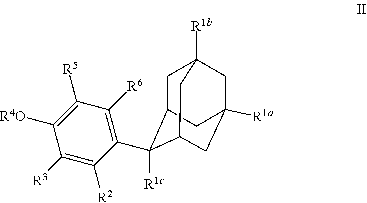
wherein:
R^{1a} and R^{1b} are independently selected from hydrogen, hydroxyl, carboxy alkyl ester, and hydroxy alkyl, optionally with the proviso that R^{1a} and R^{1b} are not the same;
R^{1c} is selected from hydrogen and hydroxyl;
R², R³, R⁵, and R⁶ are independently selected from hydrogen, deuterium, and halogen; and
R⁴ is hydrogen or a hydroxyl protecting group.

21.-23. (canceled)

24. The compound of claim 20, wherein R^{1c} is hydroxyl, R⁴ is a hydroxyl protecting group, R², R³, R⁵, and R⁶ are hydrogen, the compound having a Formula II(a):

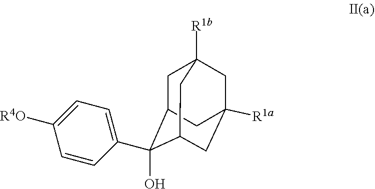

25.-28. (canceled)

29. The compound of claim 20, wherein R^{1c} is hydrogen and R⁴ is a hydroxyl protecting group, and R², R³, R⁵, and R⁶ are hydrogen, the compound having a Formula II(b):

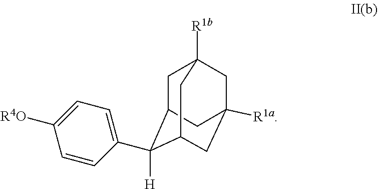

30.-31. (canceled)

32. The compound of claim 20, wherein R^{1c}, R², R³, R⁴, R⁵, and R⁶ are hydrogen, the compound having a Formula II(c):

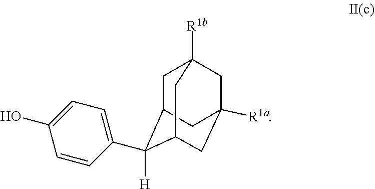

33.-34. (canceled)

35. A pharmaceutical composition comprising an effective amount of the compound of claim 20, or a pharmaceutically acceptable salt thereof, together with a pharmaceutical excipient, carrier, or diluent.

36. A method for treating a patient a disease or disorder associated with estrogen receptor β (ERβ) activity, the method comprising administering the pharmaceutical composition of claim 35.

37.-39. (canceled)

40. A compound having a Formula III: 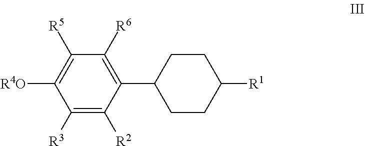
wherein R¹ is selected from hydrogen, hydroxyl, alkyl, hydroxyalkyl, and haloalkyl;
R², R³, R⁵, and R⁶ are independently selected from hydrogen, deuterium, and halogen, with the proviso that if R³, R⁵, and R⁶ are hydrogen, then R¹ is haloalkyl; and
R⁴ is hydrogen or hydroxyl protecting group.

41.-49. (canceled)

50. A pharmaceutical composition comprising an effective amount of the compound of claim 40, or a pharmaceutically acceptable salt thereof, together with a pharmaceutical excipient, carrier, or diluent.

51. A method for treating a patient a disease or disorder associated with estrogen receptor β (ERβ) activity, the method comprising administering the pharmaceutical composition of claim 50.

52.-54. (canceled)

55. A compound having a Formula IV: 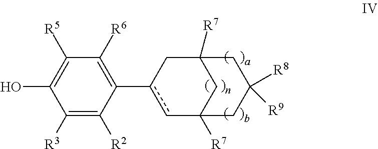
wherein R², R³, R⁵, and R⁶ are independently selected from hydrogen, deuterium, and halogen;
R⁷ is hydrogen or alkyl;
R⁸ and R⁹ are independently selected from the group consisting of hydrogen, hydroxyl, and hydroxyalkyl;
a is 0 or 1;
b is 0 or 1; and
n is 0 or 1.

56. The compound of claim 55, wherein n is 0, a and b are 1, and R⁷ is hydrogen or methyl, the compound having a Formula IV(a):

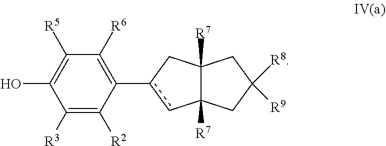

57.-58. (canceled)

59. The compound of claim 55, wherein a, b, and n are 1, and R⁷ is hydrogen, the compound having a Formula IV(b):

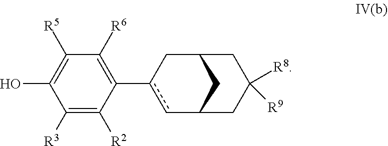

60. (canceled)

61. The compound of claim 55, wherein a, b, and n are 0, and R⁷ and R⁸ are hydrogen, and R⁹ is hydroxymethyl, the compound having a Formula IV(c):

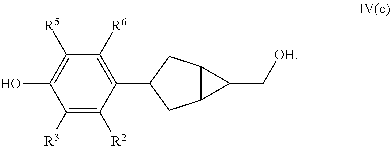

62. A pharmaceutical composition comprising an effective amount of the compound of claim 55, or a pharmaceutically acceptable salt thereof, together with a pharmaceutical excipient, carrier, or diluent.

63. A method for treating a patient a disease or disorder associated with estrogen receptor β (ERβ) activity, the method comprising administering the pharmaceutical composition of claim 62.

64.-66. (canceled)
